(19) United States
(12) Patent Application Publication
(10) Pub. No.: US 2015/0292033 A1
(43) Pub. Date: Oct. 15, 2015

(54) METHOD OF DETERMINING CANCER PROGNOSIS

(71) Applicants: Dana-Farber Cancer Institute, Inc., Boston, MA (US); Children's Medical Center Corporation, Boston, MA (US); Brigham and Women's Hospital, Boston, MA (US); The Technical University of Denmark, Kongens Lyngby (DK)

(72) Inventors: Zhigang C. Wang, Newton, MA (US); James Dirk Iglehart, Boston, MA (US); Andrea L. Richardson, Chestnut Hill, MA (US); Zoltan Szallasi, Boston, MA (US); Nicolai Juul Birbak, Copenhagen (DK); Ursula Matulonis, Lexington, MA (US)

(21) Appl. No.: 14/682,545

(22) Filed: Apr. 9, 2015

Related U.S. Application Data
(60) Provisional application No. 61/977,832, filed on Apr. 10, 2014.

(51) Int. Cl. C12Q 1/68

(52) U.S. Cl. CPC C12Q 1/6886; C12Q 2600/156; C12Q 2600/118

(57) ABSTRACT

Provided is a method of predicting the prognosis of a patient with ovarian cancer by determining the total number of somatic exome mutations per genome (Nmut) and status of the BRCA1 and/or BRCA2 in the subject.

### CROSS REFERENCE TO RELATED APPLICATION

This application claims priority to U.S. Ser. No. 61/977,832, filed on Apr. 10, 2014, the contents of which are hereby incorporated by reference in their entirety.

### FIELD OF THE INVENTION

The invention relates generally to cancer and more particularly to methods for predicting the prognosis of subjects with ovarian cancer.

### BACKGROUND OF THE INVENTION

Ovarian cancers carrying BRCA1 and BRCA2 mutations (mBRCA) display massive chromosomal alterations and are sensitive to DNA cross-linking agents containing platinum, and to PARP inhibitors. Patients with high-grade serous ovarian cancer and who carry germline mBRCA experience a longer progression-free survival (PFS) and better overall survival (OS) than non-carriers. Therefore, BRCA1 and BRCA2 may be considered biomarkers that predict response to platinum-containing chemotherapy and to PARP inhibitors. However, in previous studies 15-18% of BRCA-associated ovarian cancers responded poorly to platinum-based chemotherapy regimens, and either recurred or progressed shortly after initial surgery and chemotherapy.

### SUMMARY OF THE INVENTION

In one aspect, the invention provides a method for determining the prognosis of a subject with ovarian cancer. The method includes obtaining a cell sample from the subject and determining the total mutation burden of the sample, e.g., by determining the number of mutations in the exome of the tumor sample. The method additionally includes determining whether the BRCA1 gene and/or BRCA2 gene is mutant or wild-type in the cells to determine a BRCA1 and/or BRCA2 status for the subject. A high tumor mutation burden and a mutation in either a BRCA1 gene or BRCA2 gene indicate the subject has a better prognosis than a subject with a low tumor mutation burden.

In some embodiments, the tumor mutation burden is compared to a reference tumor mutation burden sample for a subject population whose prognostic status is known.

In some embodiments, the ovarian cancer is a serous ovarian cancer, e.g., a high grade serous cancer.

In some embodiments, the cell sample contains or is suspected of containing ovarian cancer cells.

In some embodiments, a high tumor mutation burden indicates a longer progression-free survival (PFS), a longer overall survival (OS), or both.

In some embodiments, the total mutation burden comprises single-base substitution mutations.

In some embodiments, the method comprises determining the BRCA1 status and/or the BRCA2 status of the subject (e.g., wild-type or mutant).

In some embodiments, the BRCA1 mutation and/or or BRCA2 mutation is a truncating mutation.

In some embodiments, the BRCA1 mutation and/or BRCA2 mutation is a missense mutation.

In some embodiments, the subject has had surgery to remove an ovarian tumor.

In some embodiments, the subject is classified as having a high tumor mutation burden at an Nmut of 60 or higher.

In some embodiments, the method further comprises selecting and administering a therapeutic agent or agents based on the tumor mutation burden and BRCA1/BRCA2 status.

In some embodiments, the method further comprises administering a platinum agent and a taxane if the subject has a high tumor mutation burden and a mutation in either a BRCA1 gene or BRCA2 gene.

In some embodiments, the platinum agent is carboplatin, cisplatin, or oxaliplatin.

In some embodiments, the taxane is docetaxel or paclitaxel, or a derivative or analog thereof.

In some embodiments, the method further includes creating a record indicating the subject is likely to respond to the treatment for a longer or shorter duration of time based on the BRCA1 or BRCA2 genotype and total mutation burden.

The record can be created, e.g., on a tangible medium such as a computer readable medium.

In another aspect, the invention provides a method for determining the prognosis of a subject who has had surgery to remove an ovarian tumor. The method includes obtaining a cell sample from the subject. The tumor mutation burden and status of the BRCA1 gene and/or BRCA2 gene is determined. A high tumor mutation burden and a mutation in either a BRCA1 gene or BRCA2 gene indicates that the subject has a better prognosis than a subject with a low tumor mutation burden.

In a still further aspect, the invention provides a method of diagnosing a sub-type of ovarian cancer by obtaining a cell sample from the subject. The method includes determining the tumor mutation burden of cells in the tissue sample and determining whether the BRCA1 gene or BRCA2 gene is mutant or wild-type in the cells to determine a BRCA1 and BRCA2 status for the subject. The ovarian cancer is classified as a serous ovarian cancer if the cell sample has a high tumor mutation burden and a mutation in either a BRCA1 gene or BRCA2 gene.

In another aspect, the invention provides a method for screening for a candidate agent for treating ovarian cancer. The method includes providing a cell comprising a genome with a high tumor mutation burden and a mutation in either a BRCA1 or BRCA2 gene, contacting the cell with a putative therapeutic agent, and determining whether the tumor mutation burden decreases in the cell or whether the BRCA1 gene or BRCA2 gene reverts to wild-type. A decrease in the tumor mutation burden or a reversion to a wild-type BRCA1 or BRCA2 indicates the test agent is a candidate agent for treating ovarian cancer.

In some embodiments, the candidate agent is a PARD inhibitor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention are apparent from the following description, and from the claims.

### DESCRIPTION OF DRAWINGS

FIGS. 1A-D are graphs showing the total number of exome mutations (Nmut) and clinical outcome in high-grade serous ovarian cancer. All patients received platinum and most also received taxanes.

FIG. 1A: Tumors were separated into Nmut high and low groups defined by the median Nmut across the whole cohort and compared to the rate of chemotherapy resistance. The significance of the differences was determined by Fisher's exact test.

FIG. 1B: The number of mutations (Nmut) for each tumor was compared in chemotherapy resistant and sensitive patients and is shown by dot plots. Median and 25-75 percentiles are indicated by horizontal lines. P-value is derived from the Wilcoxon rank-sum test.

FIG. 1C Kaplan-Meier analysis compared the progression-free survival (PFS) and D) overall survival (OS) between patients with high and low tumor Nmut. Patients that were progression-free or still alive at the time of last follow-up were censored (+). Numbers of patients at risk at each interval are given below the graphs. P-values are obtained by Log-rank test.

FIGS. 2A-2F are graphs showing the total number of exome mutations (Nmut) and clinical outcome in high-grade serous ovarian cancer with germline or somatic mutations in BRCA1 or BRCA2 (mBRCA) or with wild-type BRCA1 and BRCA2 (wtBRCA).

FIG. 2A shows Nmut in tumors with mBRCA. Chemotherapy resistant and sensitive ovarian cancers are shown by dot plots. P-value is derived from the Wilcoxon rank-sum test.

FIG. 2B shows Nmut in tumors with wtBRCA. Chemotherapy resistant and sensitive tumors are shown with dot plots of each tumor as in FIG. 1. Median and 25-75 percentiles are indicated by horizontal lines. P-value is derived from Wilcoxon rank-sum test.

FIG. 2C and FIG. 2D show Kaplan-Meier analysis comparing progression-free survival (PFS) (FIG. 2C) and overall survival (OS) (FIG. 2D) between patients with high and low Nmut in their mBRCA-associated tumors.

FIG. 2E and FIG. 2F show Kaplan-Meier analysis comparing PFS (FIG. 2E) and OS (FIG. 2F) in patients with high and low Nmut in their wtBRCA tumors. The median for Nmut was computed from the whole cohort of 316 tumors. In Kaplan-Meier analyses, patients that were progression-free or still alive at the time of last follow-up were censored (+). Numbers of patients at risk at each interval are given below the graphs. P-values are obtained from Log-rank test.

FIGS. 3A-F are graphs showing tumor Nmut and clinical treatment outcome in ovarian cancer patients carrying BRCA germline mutations with LOH at the BRCA loci in tumors.

FIG. 3A and FIG. 3B show Kaplan-Meier analysis comparing PFS (FIG. 3A) and OS (FIG. 3B) between Nmut high and low ovarian cancers, all of which carried either a BRCA1 or BRCA2 germline mutation with LOH at the corresponding BRCA locus.

FIG. 3C and FIG. 3D show results of Kaplan-Meier analysis comparing individual BRCA1 and BRCA2 mutation carrier groups comparing PFS (FIG. 3C) and OS (FIG. 3D).

FIG. 3E and FIG. 3F show results of Kaplan-Meier analysis in patients with BRCA2-associated tumors comparing PFS (FIG. 3E) and OS (FIG. 3F). Nmut high and low are defined as a value above or below median Nmut of all mBRCA-associated tumors. Numbers of patients at risk at each interval are given below the graphs. P-values are calculated by log-rank test.

FIG. 4A and FIG. 4B show the results of Kaplan-Meier analysis comparing PFS (FIG. 4A), and OS (FIG. 4B) between tumor Nmut high and low in patients with wtBRCA tumors and no residual disease after debulking surgery. Numbers of patients at risk at each interval are given below the graphs. P-values are obtained from Log-rank test.

FIG. 5A shows the total number of exome mutations (Nmut) in high-grade serous ovarian cancer carrying wtBRCA or mutated BRCA1/2 genes(s) (mBRCA). The tumor Nmut is presented by dot plots. Median and 25-75 percentiles are indicated by horizontal lines. P-value is derived from Wilcoxon rank-sum test.

FIG. 5B and FIG. 5C show the results obtained when tumors were separated into Nmut high and low groups defined by the median Nmut across the whole cohort and compared to the rate of chemotherapy resistance for mBRCA (FIG. 5B) and wtBRCA (FIG. 5C). The significance of the differences was determined by Fisher's exact test. OR: Odds Ratio. Confidence intervals are shown in brackets.

FIGS. 6A-6C are graphs showing the relationship between Nmut and survival in mBRCA cases based on germline or somatic origin of the BRCA1/2 mutation.

FIG. 6A shows the total number of exome mutations (Nmut) in high-grade serous ovarian cancer carrying mutated BRCA1/2 genes(s) of either germline or somatic origin. The tumor Nmut is presented by dot plots. Median and 25-75 percentiles are indicated by horizontal lines. P-value is derived from Wilcoxon rank-sum test.

FIGS. 6B and 6C are graphs showing the results of Kaplan-Meier analysis comparing PFS (FIG. 6B) and OS (FIG. 6C) between serous ovarian cancer patients with either germline or somatic mBRCA.

FIGS. 7A-B are graphs showing the position of mutations in BRCA1 (FIG. 7A) and BRCA2 (FIG. 7B) proteins by amino acid number, and their association with Nmut shows BRCA1 and BRCA2, with the domains of BRCA1 and BRCA2 proteins shown. The Y-axis shows for each mBRCA tumor Nmut, with the corresponding position of the BRCA1/2 mutation indicated on the X-axis. Germline mutations are indicated in blue, and somatic mtuations are indicated in red. Missense mutaitons are shown as diamonds.

FIGS. 7C and 7D show Nmut by grouping the locations of BRCA mutiations according to relevant regions in BRCA1 and BRCA2, respectively. Dotted lines on A) and B) show the exact grouping cut-offs. P-values comparing Nmut by location is determined by a Kruskal-Wallis test.

FIG. 8 is a graphical representation showing Nmut by BRCA1/2 mutations status, and by BRCA1 or RAD51C methylation status. P-value is based on a Wilcoxon test, and compares each group to wtBRCA independently.

FIGS. 9A-9C are graphical representations showing correlation of tumor Nmut with patient age at the time of diagnosis for germline BRCA1/2 mutation carriers (FIG. 9A), somatic BRCA1/2 mutations (FIG. 9B) or wtBRCA (FIG. 9C) tumors. Correlation between age and Nmut is determined by Spearman's rank correlation coefficient.

FIGS. 10A and 10B are graphical representations showing the correlation between tumor Nmut and the fraction of the genome with LOH (FLOH).

FIGS. 10C and 10D are graphical representations showing the correlation between tumor Nmut and the number of chromosome arms with telomeric allelic imbalance events (NtAI). BRCA genotype (mBRCA and wtBRCA) are indicated above each panel.

FIGS. 11A-D show the influence of post-surgery residual disease on progression-free and overall survival in ovarian cancer using Kaplan-Meier analysis to compare patients with to patients without residual disease in mBRCA tumors (FIGS. 11A and 11B) and wtBRCA (FIGS. 11C and 11D) tumors.

FIGS. 12A-D show the results of tumor Nmut and clinical treatment outcome in ovarian cancer patients with mBRCA tumors and residual disease or no residual disease. A) and B) Kaplan-Meier analysis compared PFS and OS between high and low Nmut in ovarian cancer patients with mBRCA and no residual disease following debulking surgery. C) and D) PFS and OS between high and low Nmut in ovarian cancer patients with mBRCA and residual disease following debulking surgery. High and low Nmut is defined by median Nmut of all mBRCA cases.

### DETAILED DESCRIPTION OF THE INVENTION

We used whole exome sequencing data from TCGA to enumerate somatic mutations and compared this to chemotherapy sensitivity, progression free survival (PFS) and overall survival (OS) in patients with ovarian cancer. A significant association between the total number of somatic exome mutations per genome (Nmut) and patient outcomes was observed in patients whose ovarian cancers possessed mutations in BRCA1 and BRCA2.

High-grade serous ovarian cancer in carriers of BRCA1 or BRCA2 has a better prognosis than the same disease in non-carriers, and may be more sensitive to cisplatin-based chemotherapy or to PARP inhibitors that target DNA repair. However, within the group of women with somatic or inherited mutations in BRCA1 or BRCA2, some patients will still have poor outcomes. There are currently no markers of treatment outcome in patients with mBRCA-associated ovarian cancer. Possible markers might include impaired apoptosis, multi-drug resistance and DNA repair proficiency. The present study sought to correlate whole-exome mutation burden in tumor tissue (Nmut) to treatment outcome in ovarian cancer patients, and to examine this relationship in patients with BRCA1 and BRCA2 mutations in their ovarian tumors.

The most remarkable association of Nmut with treatment response and outcome was seen within the subset of patients with mBRCA-associated tumors. A substantial proportion of patients with mBRCA-associated ovarian cancer but low Nmut experienced a relatively poor treatment outcome, and similar to patients with wtBRCA ovarian cancer. However, for women whose cancers were mBRCA-associated and had a high tumor Nmut, their outcome was remarkably good. This was true for both BRCA1 and BRCA2 mutations, both germline and somatic mutations, and for tumors with LOH at the corresponding locus. In patients with mBRCA-associated cancers and no residual disease after initial surgery, those with high Nmut had especially good outcomes. In fact, long survival in high-grade serous ovarian cancer, when it is observed, may be attributable to mutation in either BRCA1 or BRCA2 when these genotypes are coupled with a high tumor Nmut. Nmut is a candidate genomic marker for predicting treatment outcome in patients with mBRCA-associated ovarian cancer. The association of Nmut and outcome may reflect the degree of deficiency in BRCA1- or BRCA2-mediated DNA repair pathway(s), or the result of compensation for the deficiency by alternative mechanisms. However, all of the patients in the TCGA cohort received platinum-based chemotherapy, and the beneficial effect of a BRCA1 or BRCA2 deficiency on OS may be due to improved treatment response, or due to the less lethal potential of mBRCA-associated cancers.

In our analysis of TCGA data, BRCA1 mutation-associated ovarian cancer had a better outcome when coupled with a high tumor Nmut. In addition, BRCA1 mutation-associated cancer that lost the wild-type BRCA1 allele had a better outcome than ovarian cancer with only wild-type BRCA1 (data not shown). It is unclear why BRCA1 methylation, even coupled with high Nmut, does not translate into the same survival benefit seen in ovarian cancer with BRCA mutations and high Nmut. BRCA1 methylation is associated with a significant decrease of BRCA1 transcript levels, higher levels of genome-wide LOH and, in this study, higher mutation burden. Under selection of platinum treatment, it is possible BRCA1 methylation may be reversible, and lead to the restoration of BRCA1 expression. In breast cancer xenografts, therapy resistant triple-negative cancer lost BRCA1 promoter methylation and re-expressed the BRCA1 protein. The epigenetic co-inactivation of other gene(s), for instance in pro-apoptotic pathway(s), is a possibility that could explain the worse outcome of patients with BRCA1 methylation compared to those with BRCA1 mutation. These possibilities remain open to future studies.

Whole genome sequencing in breast cancer identified a characteristic distribution of single nucleotide mutations with an increased overall mutation burden in both BRCA1- and BRCA2-associated tumors. All possible nucleotide substitutions were seen within 96 possible trinucleotide sequence contexts without predominant patterns of particular trinucleotides, which was a characteristic signature of both BRCA1- and BRCA2-associated breast cancers. This characteristic appears consistent with loss of a key mechanism(s) for error-free DNA repair in addition to homologous recombination (HR), or activation of an error-prone DNA replication process.

Other lines of evidence show differences between BRCA1 and BRCA2 mutation-associated ovarian cancers. These differences include relatively earlier onset in BRCA1 than BRCA2 germline mutation carriers, and a relatively better survival in patients with BRCA2 than BRCA1 mutation-associated tumors in comparison to that in patients with wtBRCA-associated ovarian cancer. Our results show the same associations between tumor Nmut and treatment outcome in both BRCA1- and BRCA2-associated ovarian cancers. This observation is consistent with similar signatures of mutational processes in breast and ovarian cancers from patients with either BRCA1 or BRCA2 germline mutations. There are other well-recognized similarities between BRCA1- and BRCA2-associated diseases. These similarities include HR-mediated DNA repair deficiencies, sensitivity to DNA damaging agents and PARP inhibitors, and reversion mutation-associated treatment resistance.

A low mutation burden in tumors with either a homozygous BRCA1 or BRCA2 damaging mutation and LOH at the corresponding BRCA locus may be explained by activation of alternative mechanism(s) capable of bypassing the defect and restoring error-free DNA repair. Our knowledge of bypass pathways of repair is limited. Alternative activation of HR by concomitant loss of 53BP1 in BRCA1-deficient cells may restore resistance to PARP inhibitors, but does not change the sensitivity to cisplatin. Reversion mutation of BRCA1/2 genes in recurrent disease may result in resistance to platinum chemotherapy and PARP inhibitors, but is rarely found in the primary disease.

Prognosing Survival in a Subject with Ovarian Cancer

Obtaining Cell Samples

Cell samples in can be obtained from cancerous and non-cancerous using methods known in the art. For example, surgical procedures or needle biopsy aspiration can be used to collect cancerous samples from a subject. In some embodiments, it is important to enrich and/or purify the cancerous tissue and/or cell samples from the non-cancerous tissue and/or cell samples. In other embodiments, the cancerous tissue and/or cell samples can then be microdissected to reduce amount of normal tissue contamination prior to extraction of genomic nucleic acid or pre-RNA for use in the methods of the invention. In still another embodiment, the cancerous tissue and/or cell samples are enriched for cancer cells by at least 50%, 75%, 76%, 90%, 95%, 96%, 97%, 98%, 99%, or more or any range in between, in cancer cell content. Enrichment can be performed using, e.g., needle microdissection, laser microdissection, fluorescence activated cell sorting, and immunological cell sorting. In one embodiment, an automated machine performs the hyperproliferative cell enrichment to transform the biological sample into a purified form enriched for the presence of hyperproliferative cells.

Cells and/or nucleic acid samples from non-cancerous cells of a subject can also be obtained with surgery or aspiration.

If desired, the Nmut determined for a cell sample is compared to the Nmut of a reference cell sample from a subject or subjects whose ovarian cancer survival status is known. In one embodiment, cell and/nucleic acid samples used are taken from at least 1, 2, 5, 10, 20, 30, 40, 50, 100, or 200 different individuals.

Determining the Tumor Mutation Burden

Tumor mutation burden is determined by any sequencing method that is used to determine the coding regions (“exome”) of a tumor genome. One suitable method is measuring exome mutations as described in Bell et al., Nature 474: 609-615 2011. Methods for determining exome mutations are also disclosed in, e.g., WO2014/018860 and WO2013/015833. Whole genome sequencing methods can also be used, provided they are informative for ovarian cancer prognosis and diagnostics along with BRCA1/BRCA2 status.

In addition to the methods for determining exome mutations disclosed in the above-references, exome mutations can be performed using sequencing methods known in the art. For example, US 2013/0040863 describes methods for determining the nucleic acid sequence of a target nucleic acid molecule, including sequencing by synthesis, sequencing by ligation or sequencing by hybridization, including for mutation detection, whole genome sequencing, and exon sequencing. If desired, various amplification methods can be used to generate larger quantities, particularly of limited nucleic acid samples, prior to sequencing.

Sequencing by synthesis (SBS) and sequencing by ligation can be performed using ePCR, as used by 454 Lifesciences (Branford, Conn.) and Roche Diagnostics (Basel, Switzerland). Nucleic acids such as genomic DNA or others of interest can be fragmented, dispersed in water/oil emulsions and diluted such that a single nucleic acid fragment is separated from others in an emulsion droplet. A bead, for example, containing multiple copies of a primer, can be used and amplification carried out such that each emulsion droplet serves as a reaction vessel for amplifying multiple copies of a single nucleic acid fragment. Other methods can be used, such as bridging PCR (Illumina, Inc.; San Diego Calif.), or polony amplification (Agencourt/Applied Biosystems). US 2009/0088327; US 2010/0028885; and US 2009/0325172, each of which is incorporated herein by reference.

Methods for manual or automated sequencing are well known in the art and include, but are not limited to, Sanger sequencing, Pyrosequencing, sequencing by hybridization, sequencing by ligation and the like. Sequencing methods can be preformed manually or using automated methods. Furthermore, the amplification methods set forth herein can be used to prepare nucleic acids for sequencing using commercially available methods such as automated Sanger sequencing (available from Applied Biosystems, Foster City, Calif.) or Pyrosequencing (available from 454 Lifesciences, Branford, Conn. and Roche Diagnostics, Basel, Switzerland); for sequencing by synthesis methods commercially available from Illumina, Inc. (San Diego, Calif.) or Helicos (Cambridge, Mass.) or sequencing by ligation methods being developed by Applied Biosystems in its Agencourt platform (see also Ronaghi et al., Science 281:363 (1998); Dressman et al., Proc. Natl. Acad. Sci. USA 100:8817-8822 (2003); Mitra et al., Proc. Natl. Acad. Sci. USA 100:55926-5931 (2003)).

A population of nucleic acids in which a primer is hybridized to each nucleic acid such that the nucleic acids form templates and modification of the primer occurs in a template directed fashion. The modification can be detected to determine the sequence of the template. For example, the primers can be modified by extension using a polymerase and extension of the primers can be monitored under conditions that allow the identity and location of particular nucleotides to be determined. For example, extension can be monitored and sequence of the template nucleic acids determined using pyrosequencing, which is described in US 2005/0130173, US 2006/0134633, U.S. Pat. No. 4,971,903; U.S. Pat. No. 6,258,568 and U.S. Pat. No. 6,210,891, each of which is incorporated herein by reference, and is also commercially available. Extension can also be monitored according to addition of labeled nucleotide analogs by a polymerase, using methods described, for example, in U.S. Pat. No. 4,863,849; U.S. Pat. No. 5,302,509; U.S. Pat. No. 5,763,594; U.S. Pat. No. 5,798,210; U.S. Pat. No. 6,001,566; U.S. Pat. No. 6,664,079; U.S. 2005/0037398; and U.S. Pat. No. 7,057,026, each of which is incorporated herein by reference. Polymerases useful in sequencing methods are typically polymerase enzymes derived from natural sources. It will be understood that polymerases can be modified to alter their specificity for modified nucleotides as described, for example, in WO 01/23411; U.S. Pat. No. 5,939,292; and WO 05/024010, each of which is incorporated herein by reference. Furthermore, polymerases need not be derived from biological systems. Polymerases that are useful in the invention include any agent capable of catalyzing extension of a nucleic acid primer in a manner directed by the sequence of a template to which the primer is hybridized. Typically polymerases will be protein enzymes isolated from biological systems.

Alternatively, exon sequences can be determined using sequencing by ligation as described, for example, in Shendure et al. Science 309:1728-1732 (2005); U.S. Pat. No. 5,599,675; and U.S. Pat. No. 5,750,341, each of which is incorporated herein by reference. Sequences of template nucleic acids can be determined using sequencing by hybridization methods such as those described in U.S. Pat. No. 6,090,549; U.S. Pat. No. 6,401,267 and U.S. Pat. No. 6,620,584.

If desired, exon sequence products are detected using a ligation assay such as oligonucleotide ligation assay (OLA). Detection with OLA involves the template-dependent ligation of two smaller probes into a single long probe, using a target sequence in an amplicon as the template. In a particular embodiment, a single-stranded target sequence includes a first target domain and a second target domain, which are adjacent and contiguous. A first OLA probe and a second OLA probe can be hybridized to complementary sequences of the respective target domains. The two OLA probes are then covalently attached to each other to form a modified probe. In embodiments where the probes hybridize directly adjacent to each other, covalent linkage can occur via a ligase. One or both probes can include a nucleoside having a label such as a peptide linked label. Accordingly, the presence of the ligated product can be determined by detecting the label. In particular embodiments, the ligation probes can include priming sites configured to allow amplification of the ligated probe product using primers that hybridize to the priming sites, for example, in a PCR reaction.

Alternatively, the ligation probes can be used in an extension-ligation assay wherein hybridized probes are non-contiguous and one or more nucleotides are added along with one or more agents that join the probes via the added nucleotides. Furthermore, a ligation assay or extension-ligation assay can be carried out with a single padlock probe instead of two separate ligation probes.

Typically, tumor mutation burden in a sample from a test subject is compared to tumor mutation burden in a reference sample of a cell or cells of known ovarian cancer status. The threshold for determining whether a test sample is scored positive can be altered depending on the sensitivity or specificity desired. The clinical parameters of sensitivity, specificity, negative predictive value, positive predictive value and efficiency are typically calculated using true positives, false positives, false negatives and true negatives. A “true positive” sample is a sample that is positive according to an art recognized method, which is also diagnosed as positive (high risk for early attack) according to a method of the invention. A “false positive” sample is a sample negative by an art-recognized method, which is diagnosed positive (high risk for early attack) according to a method of the invention. Similarly, a “false negative” is a sample positive for an art-recognized analysis, which is diagnosed negative according to a method of the invention. A “true negative” is a sample negative for the assessed trait by an art-recognized method, and also negative according to a method of the invention. See, for example, Mousy (Ed.), Intuitive Biostatistics New York: Oxford University Press (1995), which is incorporated herein by reference.

As used herein, the term “sensitivity” means the probability that a laboratory method is positive in the presence of the measured trait. Sensitivity is calculated as the number of true positive results divided by the sum of the true positives and false negatives. Sensitivity essentially is a measure of how well a method correctly identifies those with disease. In a method of the invention, the Nmut values can be selected such that the sensitivity of diagnosing an individual is at least about 60%, and can be, for example, at least about 50%, 65%, 70%, 75%, 80%, 85%, 90% or 95%.

As used herein, the term “specificity” means the probability that a method is negative in the absence of the measured trait. Specificity is calculated as the number of true negative results divided by the sum of the true negatives and false positives. Specificity essentially is a measure of how well a method excludes those who do not have the measured trait. The Nmut cut-off value can be selected such that, when the sensitivity is at least about 70%, the specificity of diagnosing an individual is in the range of 30-60%, for example, 35-60%, 40-60%, 45-60% or 50-60%.

The term “positive predictive value,” as used herein, is synonymous with “PPV” and means the probability that an individual diagnosed as having the measured trait actually has the disease. Positive predictive value can be calculated as the number of true positives divided by the sum of the true positives and false positives. Positive predictive value is determined by the characteristics of the diagnostic method as well as the prevalence of the disease in the population analyzed. In a method of the invention, the Nmut cut-off values can be selected such that the positive predictive value of the method in a population having a disease prevalence of 15% is at least about 5%, and can be, for example, at least about 8%, 10%, 15%, 20%, 25%, 30% or 40%.

As used herein, the term “efficiency” means the accuracy with which a method diagnoses a disease state. Efficiency is calculated as the sum of the true positives and true negatives divided by the total number of sample results and is affected by the prevalence of the trait in the population analyzed. The Nmut cut-off values can be selected such that the efficiency of a method of the invention in a patient population having a prevalence of 15% is at least about 45%, and can be, for example, at least about 50%, 55% or 60%.

For determination of the cut-off level, receiver operating characteristic (ROC) curve analysis can be used. In some embodiments, the cut-off value for the classifier can be determined as the value that provides specificity of at least 90%, at least 80% or at least 70%.

In some embodiments, the Nmut is 60 or greater, e.g., 63.5 or greater.

### Computer Implemented Embodiments

Information from tumor mutation burden assessments and BRCA1/2 status determinations can implemented in computer programs executed on programmable computers that include, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

In some embodiments, the a machine-readable storage medium can comprise a data storage material encoded with machine readable data or data arrays which, when using a machine programmed with instructions for using the data, is capable of use for a variety of purposes, such as, without limitation, subject information relating to a diagnosing a type or subtype of ovarian cancer, evaluating the effectiveness of a treatment (e.g., surgery or chemotherapy).

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein.

The health-related data management system of the invention may also be considered to be implemented as a computer-readable storage medium, configured with a computer program, where the storage medium so configured causes a computer to operate in a specific and predefined manner to perform various functions described herein.

### Diagnosing Ovarian Cancer

Also provided by the invention is method of diagnosing ovarian cancer. A cell sample can be obtained from a subject and the tumor mutation burden of the cells determined, as is the status of the BRCA1 and/or BRCA2 genes. The subject is diagnosed with ovarian cancer if the cell sample has a high tumor mutation burden and has a mutation in either a BRCA1 gene or BRCA2 gene. In some embodiments, the ovarian cancer is a serous ovarian cancer.

### Screening for Therapeutic Agents for Treating Ovarian Cancer

The methods of the invention can also used to identify therapeutic agents for treating ovarian cancer. For example, a cell sample is provided with a genome with a high tumor mutation burden and a mutation in either a BRCA1 or BRCA2 gene, and the cell is contacted with a putative therapeutic agent. Next, the cell sample is assayed to determine whether the tumor mutation burden decreases in the cell, and/or whether the BRCA1 gene or BRCA2 gene reverts to wild-type. A decrease in the tumor mutation burden or a reversion to a wild-type BRCA1 or BRCA2 indicates the test agent is a candidate agent for treating ovarian cancer. Candidate therapeutic agents can include, e.g., a poly ADP ribose polymerase (PPARP) inhibitor.

### Kits

Also provided by the invention is a kit containing reagents for determining the total mutation burden and BRCA1/2 status. The kit can include oligonucleotides suitable for this determination, along with buffers and instructions for use. Optionally, the kits include a polymerase.

The invention will be further illustrated in the following non-limiting examples. In the examples, the total number of synonymous and non-synonymous exome mutations (Nmut), and the presence of germline or somatic mutation in BRCA1 or BRCA2 (mBRCA) were extracted from whole-exome sequences of high-grade serous ovarian cancers from The Cancer Genome Atlas (TCGA). Cox regression and Kaplan-Meier methods were used to correlate Nmut with chemotherapy response and outcome. Higher Nmut correlated with a better response to chemotherapy after surgery. In patients with mBRCA-associated cancer, low Nmut was associated with shorter progression-free survival (PFS) and overall survival (OS), independent of other prognostic factors in multivariate analysis. Patients with mBRCA-associated cancers and a high Nmut had remarkably favorable PFS and OS. The association with survival was similar in cancers with either BRCA1 or BRCA2 mutations. In cancers with wild-type BRCA, tumor Nmut was associated with treatment response in patients with no residual disease after surgery. Tumor Nmut was associated with treatment response and with both PFS and OS in patients with high-grade serous ovarian cancer carrying BRCA1 or BRCA2 mutations. In the TCGA cohort, low Nmut predicted resistance to chemotherapy, and for shorter PFS and OS, while high Nmut forecasts a remarkably favorable outcome in mBRCA-associated ovarian cancer. Our observations suggest that the total mutation burden coupled with BRCA1 or BRCA2 mutations in ovarian cancer is a genomic marker of prognosis and predictor of treatment response. This marker may reflect the degree of deficiency in BRCA-mediated pathways, or the extent of compensation for the deficiency by alternative mechanisms.

### Example 1

### General Materials and Methods

### Datasets

We obtained exome sequencing data of 316 high-grade serous ovarian cancers and follow-up information from TCGA. Any sequence alteration in the ovarian tumor exome that was not present in the germline DNA sequence was called a somatic mutation and included both non-synonymous and synonymous changes. In the exome mutation data published by the TCGA consortium, a total of 19,356 somatic mutations were identified in the cohort, and most independently validated by a second assay using whole-genome amplification of a second sample from the same tumor. Mutations that were not independently validated were computationally evaluated and had a high likelihood to be true mutations as described. Based on TCGA mutation calls explained above, the total number of somatic mutations in the tumor exome (Nmut) was determined for each case (Table 2, which shows genomic and ethnic/race information of TCGA ovarian cancer cohort used in the present study.) Affymetrix SNP6 genotyping data and updated clinical information were obtained from the TCGA data portal (http://tcga-data.nci.nih.gov/tcga/, dbGaP accession no. phs000178.v5.p5, acquired 2011 Oct. 27). BRCA1 and BRCA2 gene mutation status, BRCA1 and RAD51C methylation status and ethnic/racial information were acquired from the cBIO SU2C data portal (http://cbio.mskcc.org/su2c-portal/).

### Clinical Assessment of Therapy Response

All patients underwent debulking surgery prior to platinum and taxane-based chemotherapy. The outcome of debulking surgery was the presence or absence of visible residual disease at the end of surgery; in TCGA the dimensions of residual disease were estimated. All patients received platinum-based chemotherapy after surgery. Chemotherapy resistance was defined as disease progression during first-line platinum-based chemotherapy or progression within 6 months after completion of first-line therapy. Chemotherapy sensitivity was defined as progression-free survival longer than 6 months.

### Bioinformatics Analysis

Affymetrix SNP6 array data for tumor-normal pairs were normalized using the Aroma CRMAv2 algorithm, and B-allele fraction (BAF) was adjusted using the CalMaTe and TumorBoost Aroma packages. Processed data were analyzed for LOH, allelic imbalance, copy number changes and normal cell contamination using ASCAT. Nmut was determined by counting all mutation calls for each sample reported by the TCGA consortium (Table 1). Mutations include missense, nonsense, silent, frameshift and splice variants. The median value for Nmut was determined for the cohorts and high Nmut was defined as those values above the median, and low Nmut was values equal to or below the median. Correlation was determined by the Spearman rank correlation coefficient. Statistical significance was assessed by the Wilcoxon rank-sum test for two-group comparison or by Kruskal-Wallis test for multiple-group comparison. Survival analysis was performed using Kaplan-Meier analysis and Cox regression. For Kaplan-Meier analysis, Nmut was dichotomized around its median value in study cohorts. In Cox regression, Nmut is continuous, but hazard ratio (HR) is reported per 10 mutations. The variables for multivariate analysis included Nmut, age, stage (II, III, IV), and residual disease (not visible, <1 cm, 1-2 cm, and >2 cm). All P values are 2-sided, and all bioinformatics analysis was performed in the R 2.15.2 statistical framework.

### Example 2

### Association of Mutation Burden with Chemotherapy Sensitivity and Outcome

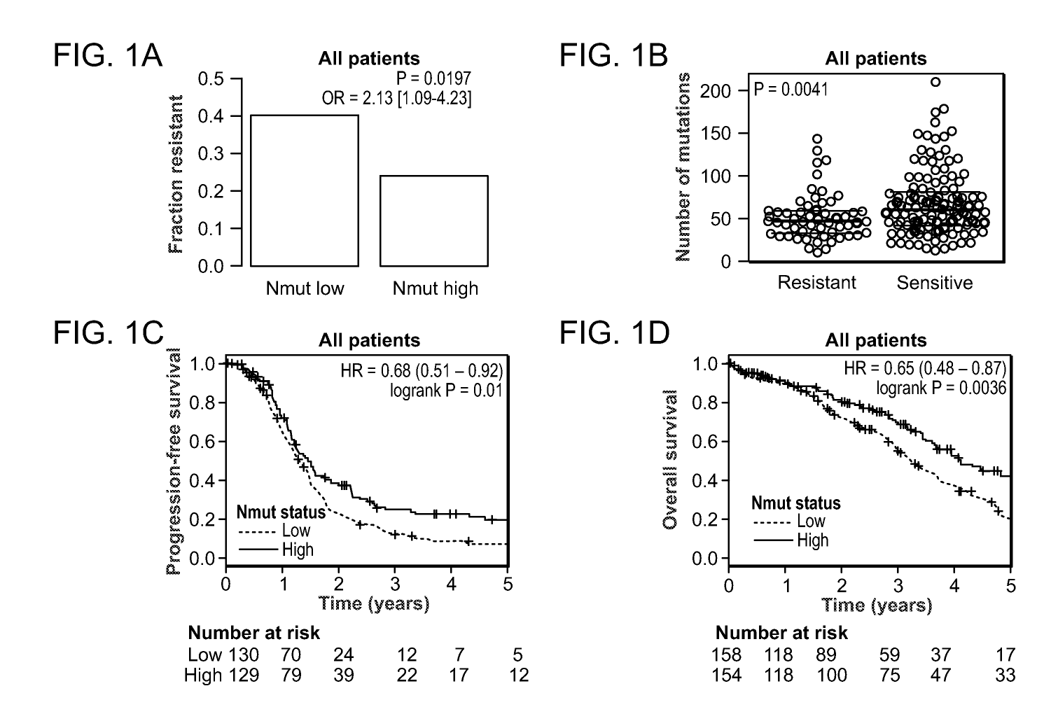

Using data from TCGA, we found that 95% of mutations in exomes of ovarian cancer are single base substitutions. Across the TCGA cohort of 316 tumors, the number of exome mutations in individual cancers (Nmut) varies widely, from 9 to 210 (median 54.5, Table 1). To determine whether Nmut is associated with chemotherapy resistance after initial surgery, we separated patients into Nmut high and low groups based on the median Nmut of the whole cohort. A higher rate of resistance to initial chemotherapy was observed in Nmut low compared to the Nmut high group (40.2 vs. 23.9%, FIG. 1A). Nmut was lower in treatment-resistant patients than sensitive patients (median 46 vs. 59, FIG. 1B). Cox regression showed a correlation between Nmut and progression-free survival (PFS) or overall survival (OS) (P=0.013 and 0.0014, respectively, Table 1). Kaplan-Meier analysis showed a significantly longer PFS and OS in the Nmut high group compared to the Nmut low group (FIGS. 1C and 1D).

### Example 3

### Effect of BRCA1 and BRCA2 on Mutation Burden and Outcome

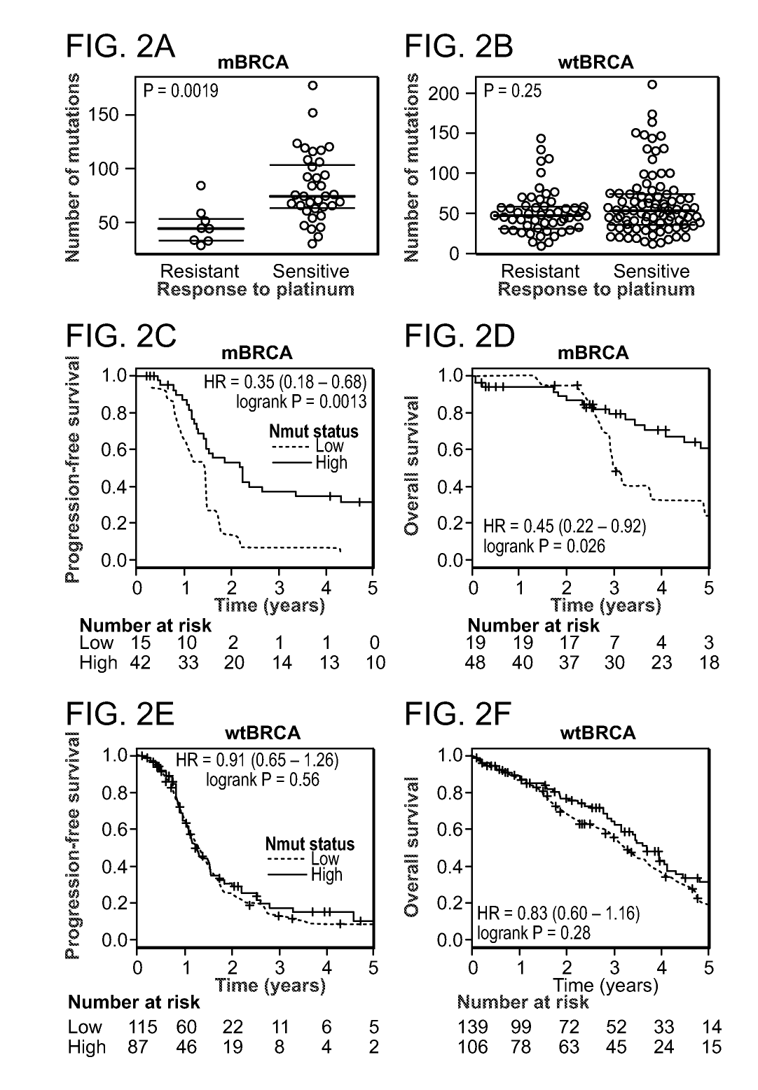

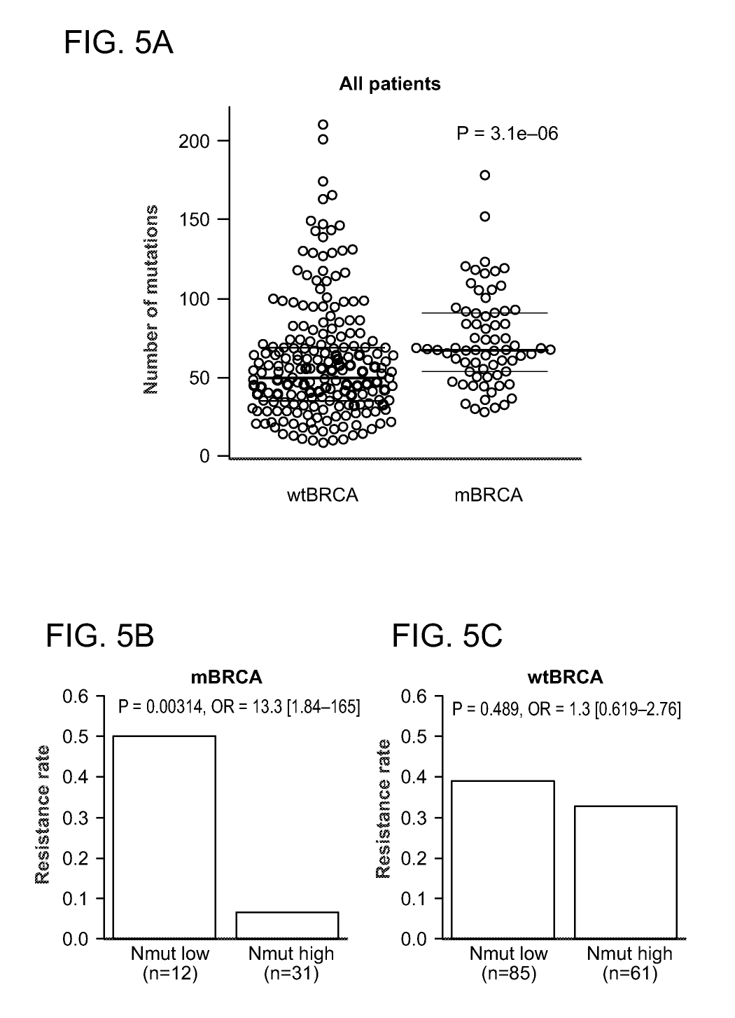

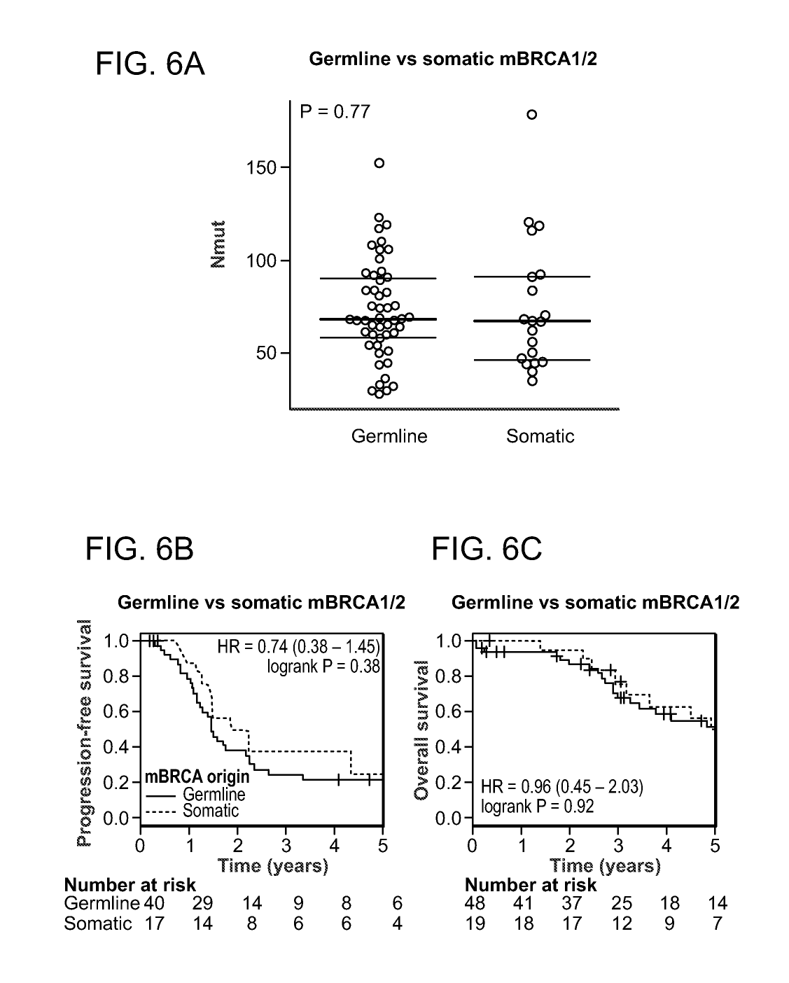

Seventy patients either carried a germline BRCA1 or BRCA2 mutation or possessed tumors bearing somatic BRCA1 or BRCA2 mutations (mBRCA). We found no differences in tumor Nmut, PFS or OS between patients with germline and tumor somatic mutations in BRCA1 and BRCA2 (FIG. 5). However, mBRCA-associated tumors possessed a higher Nmut than tumors without BRCA mutations (wtBRCA; median 67.5 vs. 49.5, FIG. 6A). We separately analyzed the subset of patients bearing mBRCA and those with wtBRCA tumors, and compared tumor Nmut between chemotherapy resistant and sensitive patients. A higher tumor Nmut predicted a higher rate of response to chemotherapy after surgery in patients with mBRCA-associated tumors, but not in those with tumors that possessed only wtBRCA (FIGS. 6B and 6C). When we investigated all patients with tumors containing mBRCA, we found a significantly higher tumor Nmut in the treatment-sensitive group versus the treatment-resistant group (median 74 vs. 44, FIG. 2A). In patients with wtBRCA tumors, there were no significant differences in Nmut between the treatment sensitive and resistant groups (median 52 vs. 47, FIG. 2B). Cox regression showed a significant correlation between tumor Nmut and PFS and OS in patients with mBRCA-associated tumors (HR=0.82, P=0.002 and HR=0.83, P=0.011, respectively), but not in patients with wtBRCA tumors (Table 1). When patients with mBRCA-associated tumors were stratified by the median Nmut of the whole cohort, patients with high tumor Nmut showed a significantly longer PFS and OS (FIGS. 2C and 2D). PFS and OS in patients with mBRCA and low tumor Nmut were shorter, similar to patients with wtBRCA tumors (FIG. 2C to 2F). In patients with wtBRCA tumors, there was no significant relationship between Nmut and PFS or OS (FIGS. 2E and 2F). Therefore, the effect of tumor Nmut on treatment response and outcome was chiefly confined to those tumors with either germline or somatic mutations in BRCA1 or BRCA2.

In univariate and multivariate analysis, stage at presentation, size of residual tumors after debulking surgery, patient age and Nmut were associated with either PFS or OS in all patients with clinical follow-up (Table 1). Strikingly, for the patients with mBRCA-associated ovarian cancer, only Nmut was significantly associated with treatment outcome in both univariate and multivariate analysis. In multivariate analysis of cancers with wtBRCA, residual disease left after initial surgery was significantly associated with both PFS and OS. Nmut and age were significantly associated with OS, but not PFS in patients with wtBRCA (Table 1). These results show Nmut is significantly associated with clinical outcome and is independent of other prognostic factors in patients with mBRCA-associated tumors.

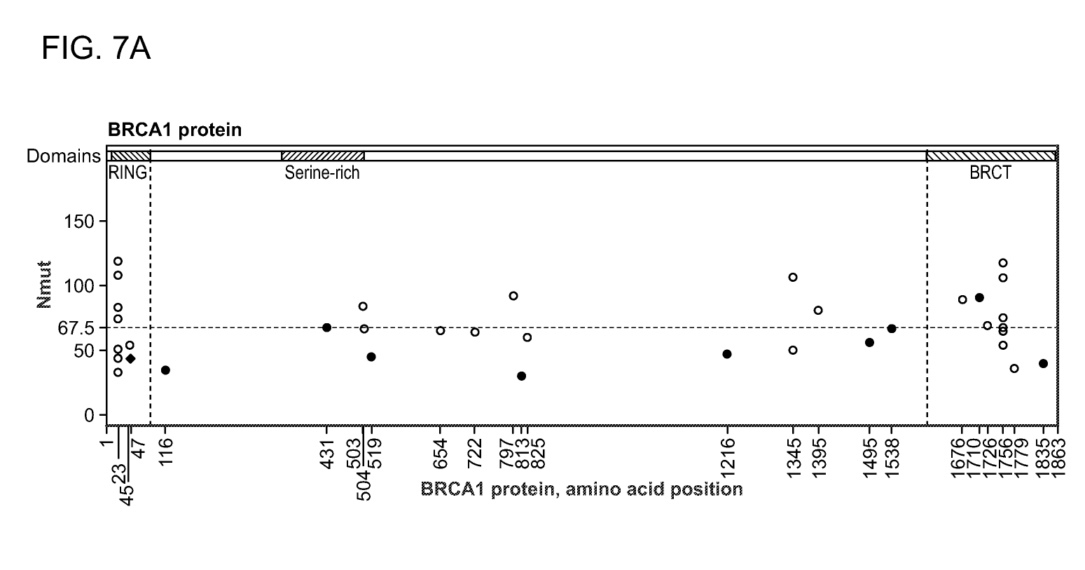

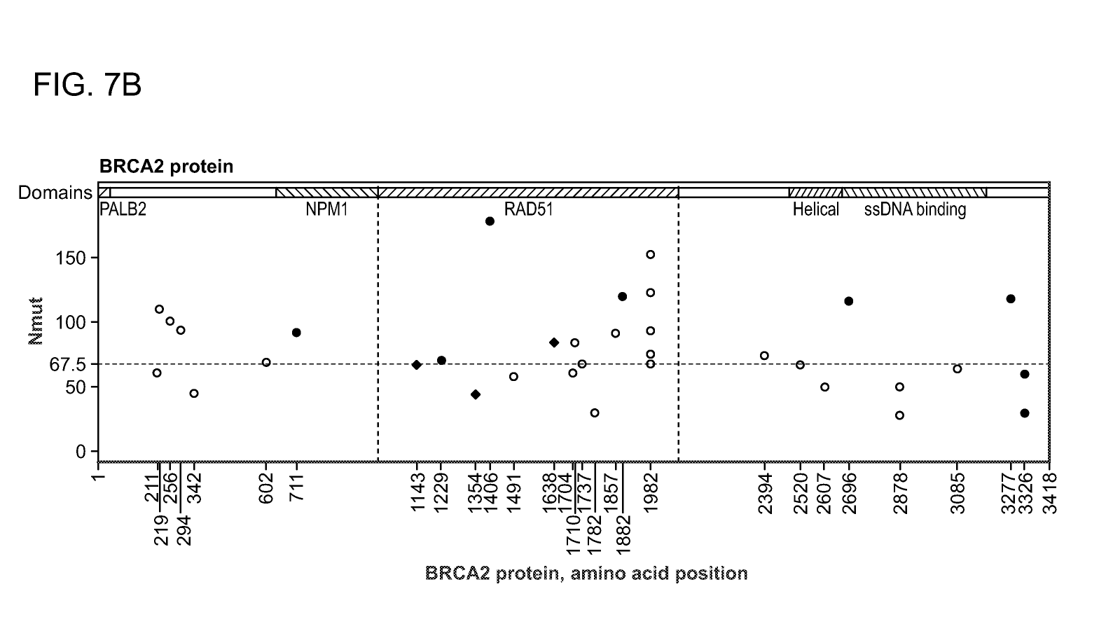

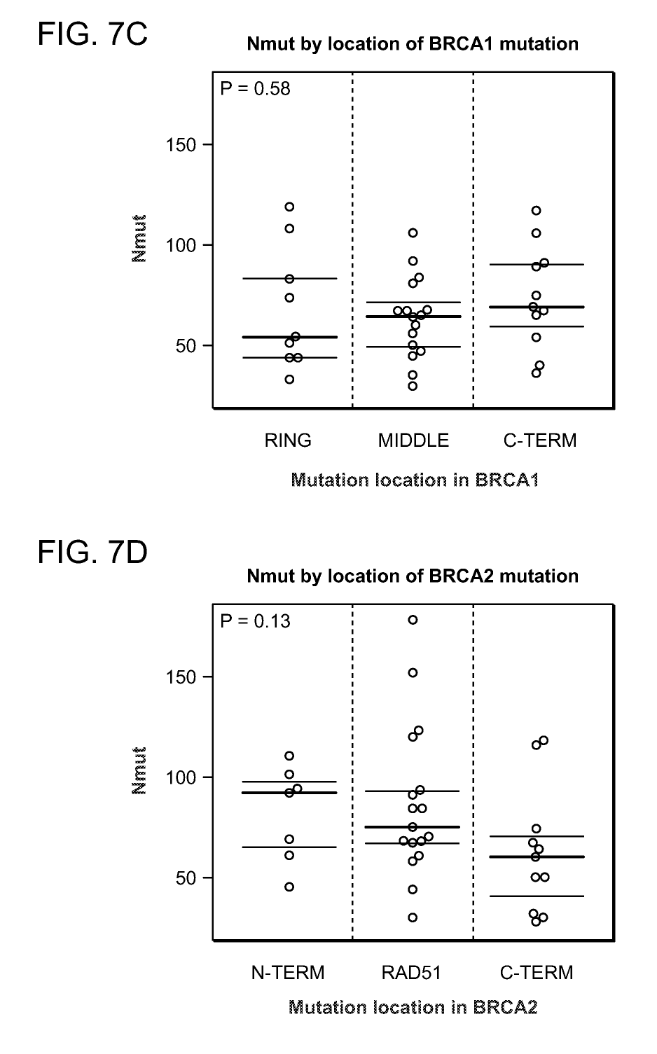

All 51 germline mutations in BRCA1 and BRCA2 were truncating mutations. Of the 21 somatic mutations in the two genes, 4 were missense and the others truncating. We examined location of the mutations in BRCA1 and BRCA2 genes for association with Nmut in tumors (FIGS. 7A and 7B). We separated BRCA mutations into ring, middle and BRCT domains of BRCA1 and N-terminal, RAD51 binding and C-terminal regions of BRCA2. Differences in Nmut among tumors with mutations in these regions of BRCA1 and BRCA2 were evaluated. No significant association was found between Nmut and mutations in different regions of BRCA1 or BRCA2 (Kruskal-Wallis test for multiple comparisons, P=0.58 and P=0.13, FIGS. 7C and 7D).

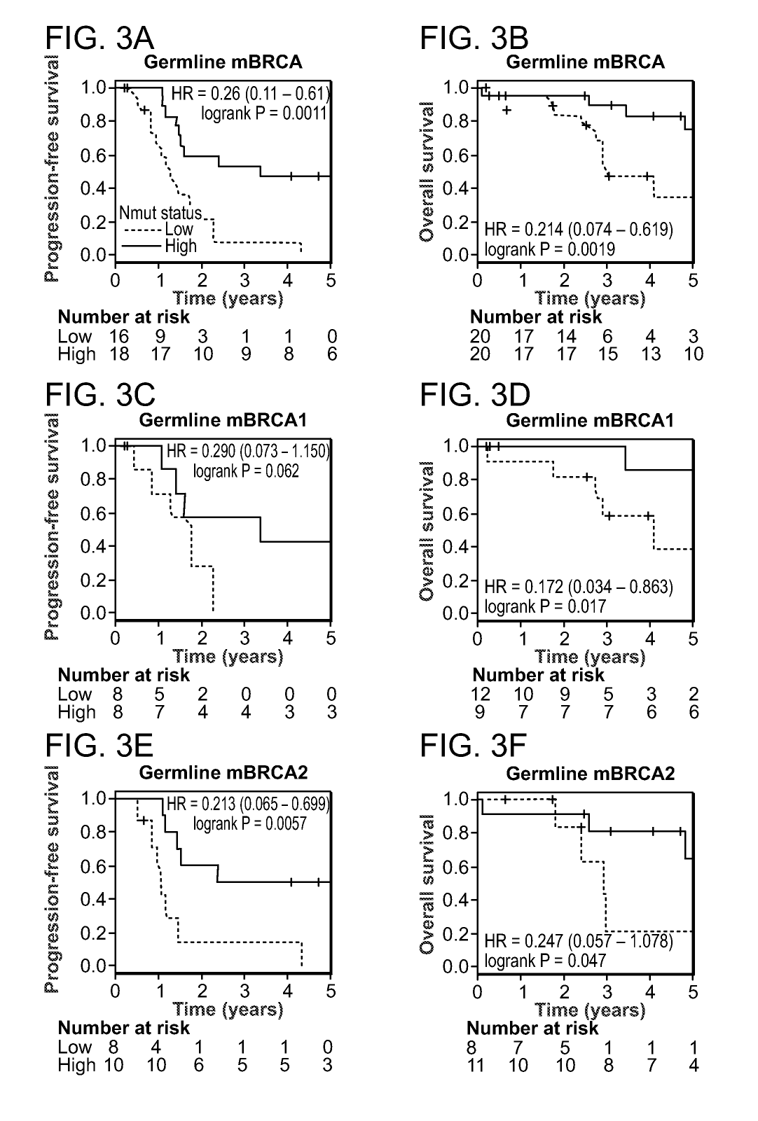

Fourteen mBRCA-associated tumors (6 somatic and 8 germline BRCA mutations) remained heterozygous at the mutated BRCA locus (Table 1 and Table 2). To avoid the influence of the wtBRCA allele, we tested for the association between tumor Nmut and clinical outcome in the subset of patients carrying BRCA germline mutations with LOH at the corresponding BRCA locus in their tumors. Cox regression revealed a significant correlation between Nmut and OS (HR=0.765, P=0.021) and a trend toward significant correlation between Nmut and PFS (HR=0.837, P=0.056). Kaplan-Meier analysis displays the remarkable differences in outcome between patients with high and low tumor mutation burden (FIGS. 3A and 3B). Despite small numbers, significant and consistent differences in PFS and OS were seen when BRCA1 and BRCA2 germline mutation carriers were evaluated separately (FIGS. 3C to 3F). These results support the conclusion that tumor Nmut is associated with both treatment response and clinical outcome within patients with inherited BRCA1 or BRCA2 mutations.

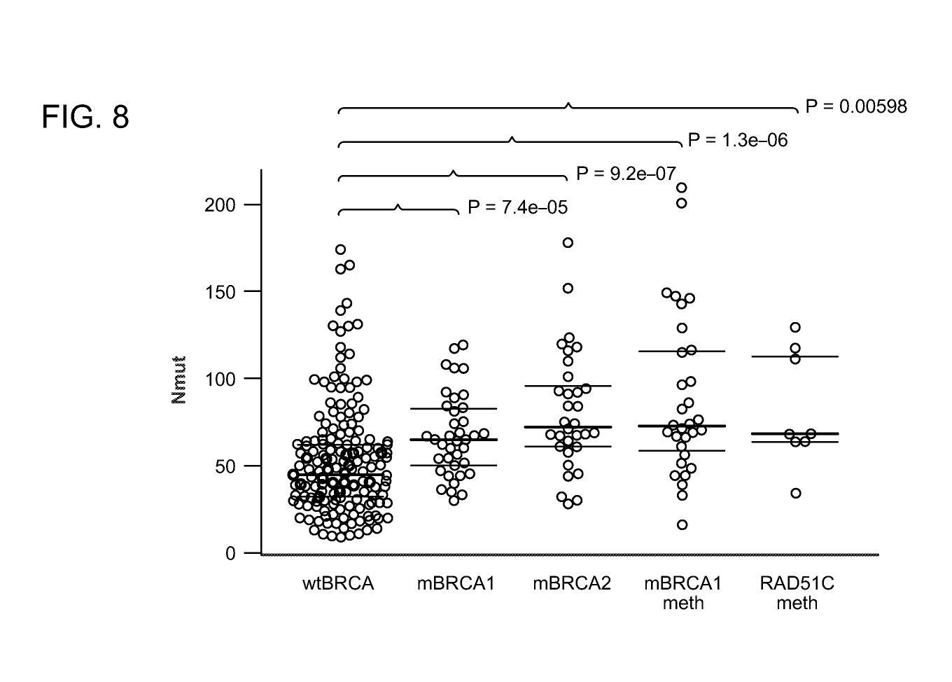

We examined Nmut in tumors with known epigenetic changes in BRCA1 (n=31) and RAD51C (n=8) in this TCGA dataset. Compared to tumors with wtBRCA and without methylation in the two genes, we observed a higher Nmut in tumors with BRCA1 or RAD51C methylation, similar to tumors with mBRCA (FIG. 8). The result suggests that epigenetic silencing in BRCA1 and RAD51C may lead to accumulation of single base substitutions. However, in agreement with previously published results, the outcomes (PFS and OS) of patients with tumors harboring BRCA1 methylation coupled with high Nmut were similar to patients whose tumors had low Nmut or wtBRCA1 (data not shown). The association between tumor Nmut and treatment outcome appears largely in cancers with BRCA1 mutation, but not in those cancers with BRCA1 epigenetic alterations.

### Example 4

### Correlation Between Nmut and Age or Chromosomal Damage

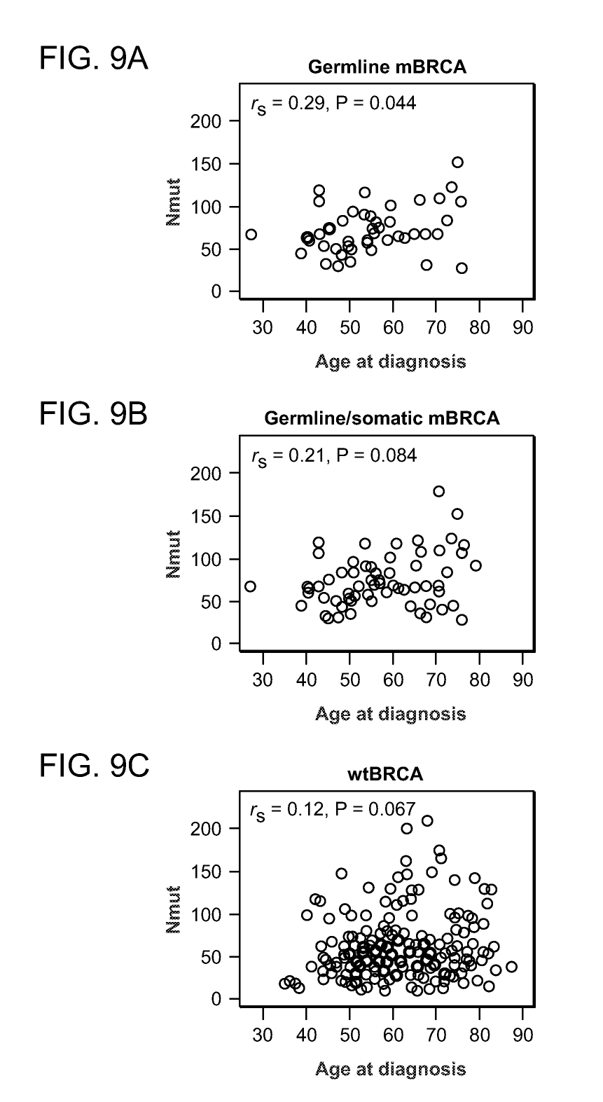

Nmut in tumors from patients with germline BRCA1 or BRCA2 mutations (BRCA mutations) increased with patient age at diagnosis (FIG. 9A). However, this relationship was lost when tumors with somatic BRCA mutations were included or those with wtBRCA were analyzed separately, (FIGS. 9B and 9C). These finding are consistent with a distinct pathogenic process in germline BRCA-associated cancers with haplo-insufficiency of BRCA function in premalignant tissue, and those cancers that acquire BRCA mutations later in their development. A similar correlation between accumulated mutations and age was reported in cancers that arise from tissues which normally replicate during life (e.g., colonic epithelium), but are not seen in cancers from tissue normally dormant (e.g., cells in the exocrine pancreas).

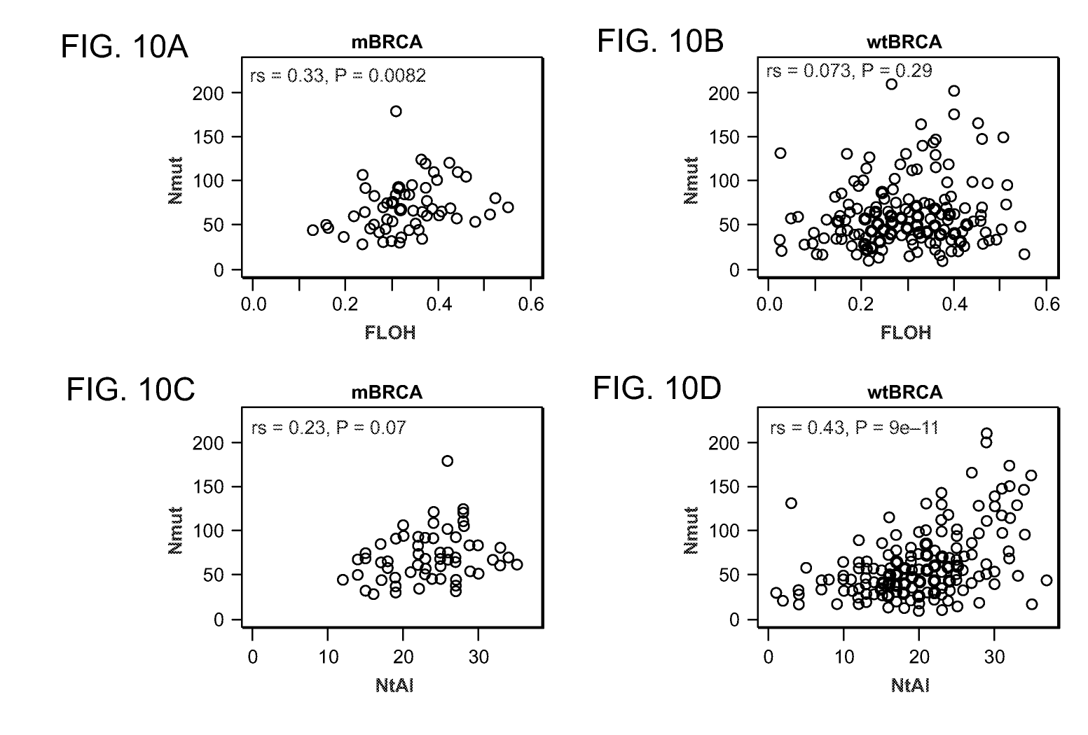

Both the fraction of LOH per genome (FLOH) and the number of episodes of telomeric allelic imbalance (NtAI) reflect the extent of tumor chromosomal damage. Using TCGA SNP6 data from the same cohort, Nmut positively correlated with FLOH and NtAI in mBRCA-associated tumors; NtAI correlated with Nmut in wtBRCA tumors (FIG. 10). The association between high mutation burden and high level of chromosomal damage suggests a link between the processes that produce or fail to repair these distinct types of DNA damage.

### Example 5

### Influence of Residual Disease on the Association of Mutation Burden and Outcome

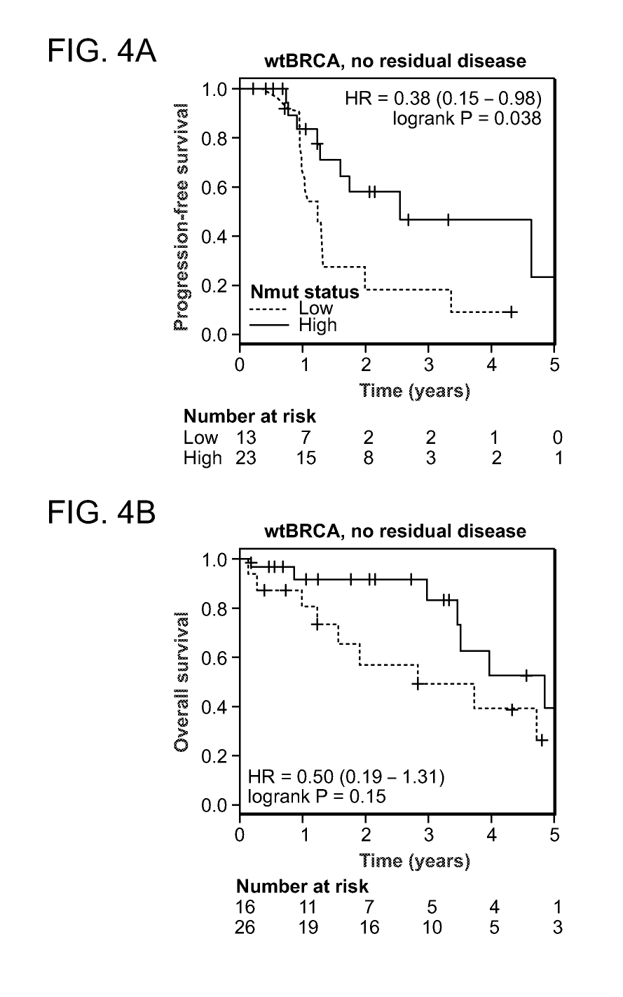

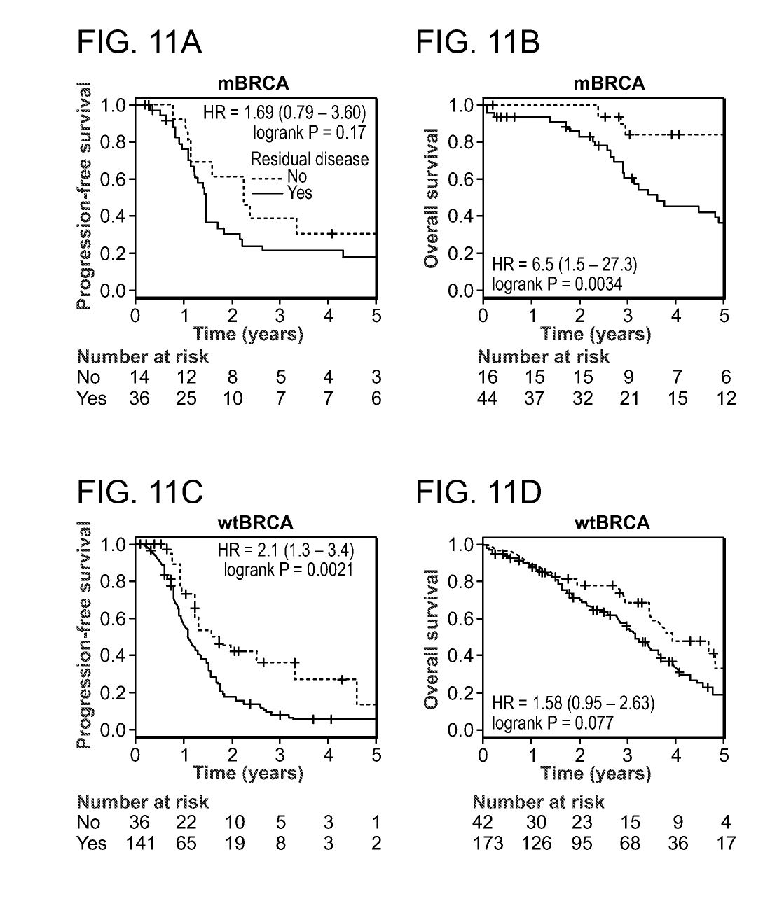

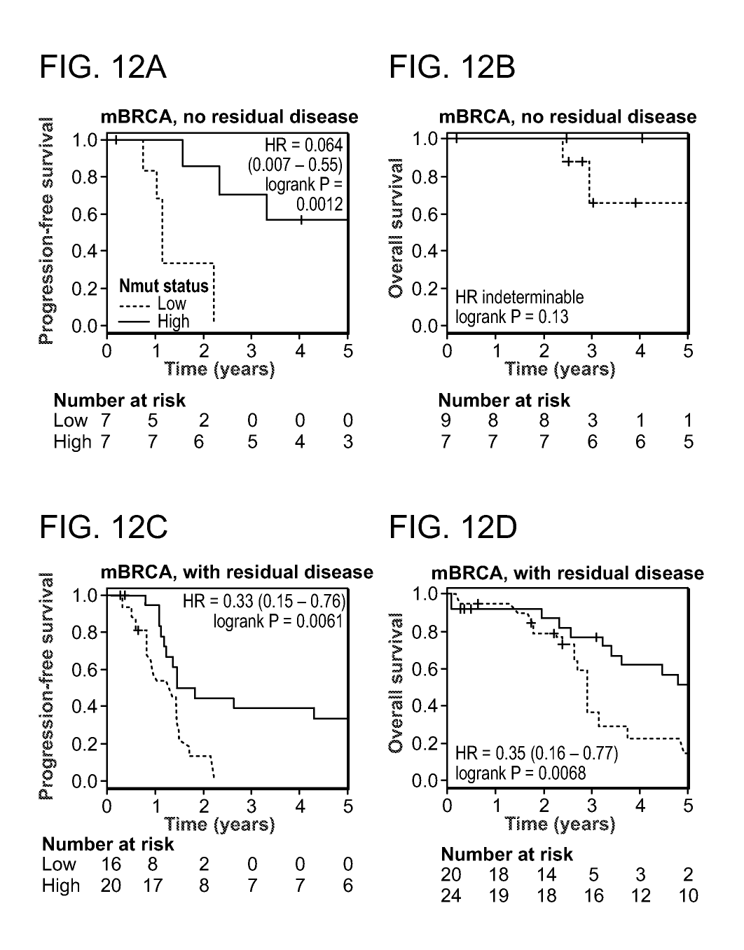

Residual disease after initial surgery is a prognostic factor in ovarian cancer and was confirmed in both mBRCA- and wtBRCA-associated ovarian cancer (FIGS. 11A-D). In patients with mBRCA-related cancers, those with a high tumor Nmut had better outcomes than those with a low tumor Nmut regardless of whether residual disease was present after initial surgery (FIGS. 12A-D). Patients with no residual disease and a high tumor Nmut had an especially favorable outcome (5 year PFS was 58% and OS was 100%; FIGS. 12A-D). In the subset of patients with wtBRCA tumors and no residual disease after surgery, high tumor Nmut predicted a longer PFS and a trend towards longer OS (FIG. 4). No such differences were found in patients with wtBRCA tumors and residual disease after surgery (data not shown). Residual disease is a powerful prognostic factor, which may mask the effect of tumor Nmut in patients with wtBRCA tumors. The result suggests Nmut is potentially associated with treatment outcome in sporadic ovarian cancer with wtBRCA and no residual disease.

### Example 6

### Treatment of a Subject with a High Nmut and at Least One BRCA1 or BRCA2 Mutation

A patient has had surgical removal of a primary ovarian cancer malignancy. Tumor tissue is submitted for “exome-sequencing”. A sample is also submitted for BRCA1 or BRCA2 testing (if the patient has not been previously undergone BRCA1 or BRCA2 testing).

The Nmut is greater than 60 and either BRCA1 or BRCA2 is positive, i.e., mutant (either the patient or the tumor). The patient receives platinum-based chemotherapy and the prognosis is very good.

### Example 7

### Prognosis Based on an Optimal Nₘᵤₜ Cutoff

Receiver operator characteristic (ROC) curve analysis is used to provide an optimal Nmut cutoff for a desired sensitivity and specificity. From ROC analysis, the conclusion is that Nmut has the ability to predict treatment response and outcome in high grade serous ovarian cancer with BRCA1/2 mutations. The prognosis is most predictive for determining sensitivity to platinum-based chemotherapy (defined by resistant/sensitive).

For tumors with BRCA1/2 mutation

| | | 3 year overall |
|---|---|---|
| | Resistant/Sensitive | survival |
| Optimal Nmut cutoff | 60 | 63.5 |
| Sensitivity | 0.8 | 0.64 |
| Specificity | 0.88 | 0.73 |
| Positive predictive value | 0.97 | 0.81 |
| (PPV) | | |
| Negative predictive value | 0.5 | 0.56 |
| (NPV) | | |

These data show that Nmut predicts treatment response and outcome in high grade serous ovarian cancer with BRCA1/2 mutations, particularly for identifying sensitivity to platinum-based chemotherapy (defined by resistant/sensitive) for tumors with a BRCA1/2 mutation.

Tumor Nmut with an optimal threshold 60 has a high value (0.97), which is predictive for good response or, sensitivity, to platinum-based chemotherapy in patients with high grade serous ovarian cancer carrying BRCA1/2 mutations. The sensitivity and specificity of the prediction are 0.8 and 0.88, respectively. The patients with tumor Nmut below the threshold are at high risk (≧50%) of being resistant to the therapy.

### REFERENCES

1. Higgins M J, Baselga J (2011) Targeted therapies for breast cancer. J Clin Invest 121: 3797-3803.
2. Johnston S R (2010) New strategies in estrogen receptor-positive breast cancer. Clin Cancer Res 16: 1979-1987.
3. Venkitaraman A R (2002) Cancer susceptibility and the functions of BRCA1 and

BRCA2. Cell 108: 171-182.
4. Richardson A L, Wang Z C, De Nicolo A, Lu X, Brown M, et al. (2006) X chromosomal abnormalities in basal-like human breast cancer. Cancer Cell 9: 121-132.
5. Walsh C S, Ogawa S, Scoles D R, Miller C W, Kawamata N, et al. (2008) Genome-wide loss of heterozygosity and uniparental disomy in BRCA1/2-associated ovarian carcinomas. Clin Cancer Res 14: 7645-7651.
6. Bolton K L, Chenevix-Trench G, Goh C, Sadetzki S, Ramus S J, et al. (2012) Association between BRCA1 and BRCA2 mutations and survival in women with invasive epithelial ovarian cancer. JAMA 307: 382-390.
7. Farmer H, McCabe N, Lord C J, Tutt A N, Johnson D A, et al. (2005) Targeting the DNA repair defect in BRCA mutant cells as a therapeutic strategy. Nature 434: 917-921.
8. Alsop K, Fereday S, Meldrum C, deFazio A, Emmanuel C, et al. (2012) BRCA mutation frequency and patterns of treatment response in BRCA mutation-positive women with ovarian cancer: a report from the Australian Ovarian Cancer Study Group. J Clin Oncol 30: 2654-2663.
9. Bell D, Berchuck A, Birrer M, Chien J, Cramer D W, et al. (2011) Integrated genomic analyses of ovarian carcinoma. Nature 474: 609-615.
10. Bowtell D D (2010) The genesis and evolution of high-grade serous ovarian cancer.

Nat Rev Cancer 10: 803-808.
11. Wang Z C, Birkbak N J, Culhane A C, Drapkin R, Fatima A, et al. (2012) Profiles of genomic instability in high-grade serous ovarian cancer predict treatment outcome. Clin Cancer Res 18: 5806-5815.
12. Birkbak N J, Wang Z C, Kim J Y, Eklund A C, Li Q, et al. (2012) Telomeric allelic imbalance indicates defective DNA repair and sensitivity to DNA-damaging agents. Cancer Discov 2: 366-375.
13. Mukhopadhyay A, Elattar A, Cerbinskaite A, Wilkinson S J, Drew Y, et al. (2010) Development of a functional assay for homologous recombination status in primary cultures of epithelial ovarian tumor and correlation with sensitivity to poly(ADP-ribose) polymerase inhibitors. Clin Cancer Res 16: 2344-2351.
14. Nik-Zainal S, Van Loo P, Wedge D C, Alexandrov L B, Greenman C D, et al. (2012)

The life history of 21 breast cancers. Cell 149: 994-1007.
15. Yang D, Khan S, Sun Y, Hess K, Shmulevich I, et al. (2011) Association of BRCA1 and BRCA2 mutations with survival, chemotherapy sensitivity, and gene mutator phenotype in patients with ovarian cancer. JAMA 306: 1557-1565.
16. Tomasetti C, Vogelstein B, Parmigiani G (2013) Half or more of the somatic mutations in cancers of self-renewing tissues originate prior to tumor initiation. Proc Natl Acad Sci USA 110: 1999-2004.
17. Silver D P, Livingston D M (2012) Mechanisms of BRCA1 tumor suppression. Cancer Discov 2: 679-684.
18. Pathania S, Nguyen J, Hill S J, Scully R, Adelmant G O, et al. (2011) BRCA1 is required for postreplication repair after UV-induced DNA damage. Mol Cell 44: 235-251.
19. Xie J, Litman R, Wang S, Peng M, Guillemette S, et al. (2010) Targeting the FANCJ-BRCA1 interaction promotes a switch from recombination to poleta-dependent bypass. Oncogene 29: 2499-2508.
20. Alexandrov L B, Nik-Zainal S, Wedge D C, Aparicio S A, Behjati S, et al. (2013) Signatures of mutational processes in human cancer. Nature 500: 415-421.
21. Dhillon K K, Swisher E M, Taniguchi T (2011) Secondary mutations of BRCA1/2 and drug resistance. Cancer Sci 102: 663-669.
22. Turner N, Tutt A, Ashworth A (2004) Hallmarks of ‘BRCAness’ in sporadic cancers.

Nat Rev Cancer 4: 814-819.
23. Bunting S F, Callen E, Wong N, Chen H T, Polato F, et al. (2010) 53BP1 inhibits homologous recombination in Brcal-deficient cells by blocking resection of DNA breaks. Cell 141: 243-254.
24. Bunting S F, Callen E, Kozak M L, Kim J M, Wong N, et al. (2012) BRCA1 functions independently of homologous recombination in DNA interstrand crosslink repair. Mol Cell 46: 125-135.
25. Fung-Kee-Fung M, Oliver T, Elit L, Oza A, Hirte H W, et al. (2007) Optimal chemotherapy treatment for women with recurrent ovarian cancer. Curr Oncol 14: 195-208.
26. Bengtsson H, Wirapati P, Speed T P (2009) A single-array preprocessing method for estimating full-resolution raw copy numbers from all Affymetrix genotyping arrays including GenomeWideSNP 5 & 6. Bioinformatics 25: 2149-2156.
27. Bengtsson H, Neuvial P, Speed T P (2010) TumorBoost: normalization of allele-specific tumor copy numbers from a single pair of tumor-normal genotyping microarrays. BMC Bioinformatics 11: 245.
28. Ortiz-Estevez M, Aramburu A, Bengtsson H, Neuvial P, Rubio A (2012) CalMaTe: a method and software to improve allele-specific copy number of SNP arrays for downstream segmentation. Bioinformatics 28: 1793-1794.
29. Van Loo P, Nordgard S H, Lingjaerde O C, Russnes H G, Rye I H, et al. (2010) Allele-specific copy number analysis of tumors. Proc Natl Acad Sci USA 107: 16910-16915.

It is to be understood that the foregoing description is intended to illustrate and not to limit the scope of the invention, which is defined by the scope of the appended claims.

Other embodiments are within the scope of the following claims.

TABLE 1
Univariate and Multivariate analysis of Nmut
and other clinical variables with PFS and OS.

| | | Univariate | | | Multivariate | | |
|---|---|---|---|---|---|---|---|
| | | HR^{a} | 95% CI^{b} | P^{c} | HR | 95% CI | P |
| All cases | | | | | | | |
| Nmut^{d} | PFS | 0.944 | (0.990- | 0.013 | 0.955 | (0.991- | 0.042 |
| | | | 0.999) | | | 1.000) | |
| | OS | 0.926 | (0.988- | 0.0014 | 0.913 | (0.986- | 0.0001 |
| | | | 0.997) | | | 0.996) | |
| Stage | PFS | 1.466 | (1.072- | 0.017 | 1.38 | (0.985- | 0.061 |
| | | | 2.005) | | | 1.935) | |
| | OS | 1.325 | (0.960- | 0.087 | 1.221 | (0.865- | 0.256 |
| | | | 1.828) | | | 1.724) | |
| Residual^{e} | PFS | 1.183 | (1.026- | 0.021 | 1.158 | (0.999- | 0.052 |
| | | | 1.365) | | | 1.342) | |
| | OS | 1.267 | (1.091- | 0.0019 | 1.245 | (1.065- | 0.006 |
| | | | 1.470) | | | 1.455) | |
| Age (yrs) | PFS | 0.995 | (0.982- | 0.492 | 0.998 | (0.984- | 0.828 |
| | | | 1.009) | | | 1.013) | |
| | OS | 1.019 | (1.005- | 0.0075 | 1.025 | (1.010- | 0.001 |
| | | | 1.033) | | | 1.040) | |
| mBRCA | | | | | | | |
| Nmut | PFS | 0.817 | (0.968- | 0.002 | 0.856 | (0.971- | 0.027 |
| | | | 0.993) | | | 0.998) | |
| | OS | 0.828 | (0.967- | 0.011 | 0.821 | (0.966- | 0.0082 |
| | | | 0.996) | | | 0.995) | |
| Stage | PFS | 1.694 | (0.745- | 0.209 | 1.415 | (0.600- | 0.428 |
| | | | 3.853) | | | 3.338) | |
| | OS | 1.304 | (0.539- | 0.555 | 1.1 | (0.396- | 0.856 |
| | | | 3.154) | | | 3.055) | |
| Residual | PFS | 0.999 | (0.723- | 0.993 | 0.979 | (0.695- | 0.904 |
| | | | 1.379) | | | 1.379) | |
| | OS | 1.362 | (0.959- | 0.084 | 1.389 | (0.961- | 0.081 |
| | | | 1.936) | | | 2.009) | |
| Age (yrs) | PFS | 0.987 | (0.960- | 0.378 | 0.999 | (0.967- | 0.928 |
| | | | 1.016) | | | 1.031) | |
| | OS | 1.017 | (0.985- | 0.301 | 1.023 | (0.990- | 0.175 |
| | | | 1.049) | | | 1.058) | |
| wtBRCA | | | | | | | |
| Nmut | PFS | 0.987 | (0.994- | 0.593 | 0.989 | (0.994- | 0.648 |
| | | | 1.003) | | | 1.004) | |
| | OS | 0.966 | (0.992- | 0.159 | 0.948 | (0.990- | 0.032 |
| | | | 1.001) | | | 1.000) | |
| Stage | PFS | 1.369 | (0.980- | 0.065 | 1.234 | (0.859- | 0.255 |
| | | | 1.913) | | | 1.772) | |
| | OS | 1.224 | (0.871- | 0.244 | 1.119 | (0.778- | 0.545 |
| | | | 1.719) | | | 1.608) | |
| Residual | PFS | 1.231 | (1.048- | 0.011 | 1.219 | (1.030- | 0.021 |
| | | | 1.447) | | | 1.443) | |
| | OS | 1.195 | (1.011- | 0.037 | 1.192 | (1.000- | 0.051 |
| | | | 1.414) | | | 1.421) | |
| Age (yrs) | PFS | 0.994 | (0.979- | 0.466 | 0.998 | (0.982- | 0.841 |
| | | | 1.010) | | | 1.015) | |
| | OS | 1.017 | (1.001- | 0.035 | 1.024 | (1.007- | 0.0051 |
| | | | 1.033) | | | 1.041) | |

^{a}Hazard ratio
^{b}95% confidence interval
^{c}P-value from Cox proportional hazard regression
^{d}HR for Nmut is expressed the ratio per 10 mutations
^{e}Residual disease left after initial surgery

TABLE 2

| | | | | | | mBRCA1 | mBRCA2 | mBRCA1 |
|---|---|---|---|---|---|---|---|---|
| Patient ID | Nmut | FLOH | NtAI | mBRCA status | mBRCA type | Germline/Somatic | Germline/Somatic | LOH status |
| TCGA-04-1331 | 92 | 0.313680774 | 23 | mBRCA | mBRCA2 | NA | S | NA |
| TCGA-04-1336 | 68 | 0.388127662 | 23 | mBRCA | mBRCA2 | NA | G | NA |
| TCGA-04-1356 | 64 | 0.365615514 | 27 | mBRCA | mBRCA1 | G | NA | Het loss |
| TCGA-04-1357 | 67 | NA | NA | mBRCA | mBRCA1 | S | NA | Diploid |
| TCGA-04-1367 | 94 | 0.343178376 | 20 | mBRCA | mBRCA2 | NA | G | NA |
| TCGA-09-1669 | 50 | 0.263534036 | 23 | mBRCA | mBRCA1 | G | NA | Het loss |
| TCGA-09-2045 | 36 | 0.195880206 | 19 | mBRCA | mBRCA1 | G | NA | Het loss |
| TCGA-09-2050 | 120 | 0.422617171 | 24 | mBRCA | mBRCA2 | NA | S | NA |
| TCGA-09-2051 | 106 | 0.457851746 | 28 | mBRCA | mBRCA1 | G | NA | Het loss |
| TCGA-10-0931 | 33 | 0.364068057 | 27 | mBRCA | mBRCA1 | G | NA | Het loss |
| TCGA-13-0726 | 74 | 0.303151605 | 15 | mBRCA | mBRCA2 | NA | G | NA |
| TCGA-13-0730 | 40 | 0.273745058 | 27 | mBRCA | mBRCA1 | S | NA | Het loss |
| TCGA-13-0761 | 56 | 0.291464001 | 23 | mBRCA | mBRCA1 | S | NA | Het loss |
| TCGA-13-0792 | 67 | 0.345873245 | 32 | mBRCA | mBRCA2 | NA | S | NA |
| TCGA-13-0793 | 64 | 0.407286543 | 17 | mBRCA | mBRCA2 | NA | G | NA |
| TCGA-13-0804 | 44 | 0.339808508 | 12 | mBRCA | mBRCA1 | S | NA | Het loss |
| TCGA-13-0883 | 65 | 0.242173814 | 18 | mBRCA | mBRCA1 | G | NA | Het loss |
| TCGA-13-0885 | 178 | 0.309876212 | 26 | mBRCA | mBRCA2 | NA | S | NA |
| TCGA-13-0886 | 68 | 0.282068826 | 15 | mBRCA | mBRCA2 | NA | G | NA |
| TCGA-13-0887 | 119 | 0.372255453 | 28 | mBRCA | mBRCA1 | G | NA | Het loss |
| TCGA-13-0890 | 70 | 0.548745045 | 27 | mBRCA | mBRCA2 | NA | S | NA |
| TCGA-13-0893 | 84 | 0.309562424 | 30 | mBRCA | mBRCA1 | G | NA | Het loss |
| TCGA-13-0900 | 101 | 0.397047001 | 26 | mBRCA | mBRCA2 | NA | G | NA |
| TCGA-13-0903 | 67 | 0.317934075 | 26 | mBRCA | mBRCA1 | G | NA | Het loss |
| TCGA-13-0913 | 91 | 0.371688465 | 24 | mBRCA | mBRCA2 | NA | G | NA |
| TCGA-13-1408 | 83 | 0.260664662 | 29 | mBRCA | mBRCA1 | G | NA | Het loss |
| TCGA-13-1481 | 116 | NA | NA | mBRCA | mBRCA2 | NA | S | NA |
| TCGA-13-1489 | 62 | 0.514140561 | 35 | mBRCA | mBRCA1 | NA | NA | NA |
| TCGA-13-1494 | 54 | 0.480046045 | 29 | mBRCA | mBRCA1 | G | NA | Het loss |
| TCGA-13-1498 | 123 | 0.364087004 | 28 | mBRCA | mBRCA2 | NA | G | NA |
| TCGA-13-1499 | 75 | 0.300834051 | 22 | mBRCA | mBRCA2 | NA | G | NA |
| TCGA-13-1512 | 60 | NA | NA | mBRCA | mBRCA1/2 | G | G | Het loss |
| TCGA-23-1026 | 30 | 0.280921997 | 27 | mBRCA | mBRCA1/2 | S | G | Het loss |
| TCGA-23-1027 | 44 | 0.357375891 | 27 | mBRCA | mBRCA1 | G | NA | Diploid |
| TCGA-23-1030 | 44 | 0.129762544 | 17 | mBRCA | mBRCA2 | NA | S | NA |
| TCGA-23-1118 | 74 | 0.290533206 | 25 | mBRCA | mBRCA1 | G | NA | Het loss |
| TCGA-23-1120 | 118 | NA | NA | mBRCA | mBRCA2 | NA | S | NA |
| TCGA-23-1122 | 117 | NA | NA | mBRCA | mBRCA1 | G | NA | Amp |
| TCGA-23-2077 | 75 | 0.375714735 | 26 | mBRCA | mBRCA1 | G | NA | Het loss |
| TCGA-23-2078 | 108 | 0.38934597 | 24 | mBRCA | mBRCA1 | G | NA | Het loss |
| TCGA-23-2079 | 51 | 0.353217818 | 30 | mBRCA | mBRCA1 | G | NA | Diploid |
| TCGA-23-2081 | 54 | 0.298817011 | 21 | mBRCA | mBRCA1 | G | NA | Het loss |
| TCGA-24-0975 | 61 | 0.375017961 | 25 | mBRCA | mBRCA2 | NA | G | NA |
| TCGA-24-1103 | 84 | 0.329128688 | 22 | mBRCA | mBRCA2 | NA | S | NA |
| TCGA-24-1417 | 61 | 0.403755291 | 33 | mBRCA | mBRCA2 | NA | G | NA |
| TCGA-24-1463 | 69 | 0.427266055 | 25 | mBRCA | mBRCA2 | NA | G | NA |
| TCGA-24-1470 | 89 | NA | NA | mBRCA | mBRCA1 | G | NA | Het loss |
| TCGA-24-1555 | 50 | 0.158944958 | 14 | mBRCA | mBRCA2 | NA | G | NA |
| TCGA-24-1562 | 32 | 0.299836384 | 15 | mBRCA | mBRCA2 | NA | G | NA |
| TCGA-24-2024 | 84 | 0.337349819 | 17 | mBRCA | mBRCA2 | NA | G | NA |
| TCGA-24-2035 | 91 | 0.314279593 | 19 | mBRCA | mBRCA1 | S | NA | Het loss |
| TCGA-24-2280 | 152 | NA | NA | mBRCA | mBRCA2 | NA | G | NA |
| TCGA-24-2288 | 110 | 0.440668446 | 28 | mBRCA | mBRCA2 | NA | G | NA |
| TCGA-24-2293 | 67 | NA | NA | mBRCA | mBRCA2 | NA | G | NA |
| TCGA-24-2298 | 81 | 0.524353559 | 33 | mBRCA | mBRCA1 | G | NA | Diploid |
| TCGA-25-1318 | 58 | 0.44198029 | 18 | mBRCA | mBRCA2 | NA | G | NA |
| TCGA-25-1625 | 35 | 0.321972969 | 22 | mBRCA | mBRCA1 | S | NA | Het loss |
| TCGA-25-1630 | 45 | 0.28739024 | 25 | mBRCA | mBRCA1 | S | NA | Het loss |
| TCGA-25-1632 | 47 | 0.163929918 | 19 | mBRCA | mBRCA1 | S | NA | Het loss |
| TCGA-25-1634 | 28 | 0.237305551 | 16 | mBRCA | mBRCA2 | NA | G | NA |
| TCGA-25-2392 | 106 | 0.236664882 | 20 | mBRCA | mBRCA1 | G | NA | Diploid |
| TCGA-25-2401 | 67 | 0.314342659 | 26 | mBRCA | mBRCA1 | G | NA | Het loss |
| TCGA-25-2404 | 45 | 0.255274288 | 24 | mBRCA | mBRCA2 | NA | G | NA |
| TCGA-29-2427 | 68 | 0.319027061 | 14 | mBRCA | mBRCA1 | S | NA | Het loss |
| TCGA-57-1582 | 69 | 0.307249372 | 34 | mBRCA | mBRCA1 | G | NA | Gain |
| TCGA-57-1584 | 30 | 0.31814723 | 19 | mBRCA | mBRCA2 | NA | G | NA |
| TCGA-59-2348 | 92 | 0.243837093 | 22 | mBRCA | mBRCA1 | G | NA | Het loss |
| TCGA-59-2351 | 93 | 0.321596934 | 27 | mBRCA | mBRCA2 | NA | G | NA |
| TCGA-61-2008 | 60 | 0.219191743 | 22 | mBRCA | mBRCA1 | G | NA | Het loss |
| TCGA-61-2109 | 65 | 0.408904657 | 27 | mBRCA | mBRCA1 | G | NA | Het loss |
| TCGA-13-1501 | 74 | 0.335106332 | 27 | wtBRCA | wtBRCA | NA | NA | NA |
| TCGA-13-0800 | 56 | 0.123366487 | 13 | wtBRCA | wtBRCA | NA | NA | NA |
| TCGA-13-0760 | 201 | 0.397278611 | 29 | wtBRCA | wtBRCA | NA | NA | NA |
| TCGA-13-0906 | 98 | 0.38427231 | 28 | wtBRCA | wtBRCA | NA | NA | NA |
| TCGA-61-2095 | 131 | 0.025217956 | 3 | wtBRCA | wtBRCA | NA | NA | NA |
| TCGA-23-1123 | 95 | 0.193634372 | 17 | wtBRCA | wtBRCA | NA | NA | NA |
| TCGA-20-0990 | 82 | 0.242974907 | 21 | wtBRCA | wtBRCA | NA | NA | NA |
| TCGA-13-0923 | 139 | 0.330796185 | 30 | wtBRCA | wtBRCA | NA | NA | NA |
| TCGA-13-1496 | 64 | 0.182275018 | 17 | wtBRCA | wtBRCA | NA | NA | NA |
| TCGA-24-2267 | 114 | 0.210332765 | 16 | wtBRCA | wtBRCA | NA | NA | NA |
| TCGA-09-2056 | 78 | 0.380598702 | 22 | wtBRCA | wtBRCA | NA | NA | NA |
| TCGA-13-1477 | 58 | 0.055746842 | 5 | wtBRCA | wtBRCA | NA | NA | NA |
| TCGA-24-1422 | 129 | 0.360159992 | 28 | wtBRCA | wtBRCA | NA | NA | NA |
| TCGA-23-1110 | 117 | 0.284891375 | 24 | wtBRCA | wtBRCA | NA | NA | NA |
| TCGA-23-1031 | 111 | 0.311438276 | 29 | wtBRCA | wtBRCA | NA | NA | NA |
| TCGA-13-0924 | 68 | 0.423351139 | 22 | wtBRCA | wtBRCA | NA | NA | NA |
| TCGA-24-1104 | 68 | 0.35653407 | 26 | wtBRCA | wtBRCA | NA | NA | NA |
| TCGA-25-2391 | 64 | 0.358260909 | 21 | wtBRCA | wtBRCA | NA | NA | NA |
| TCGA-59-2354 | 64 | 0.322069733 | 19 | wtBRCA | wtBRCA | NA | NA | NA |
| TCGA-25-2399 | 47 | 0.220333298 | 10 | wtBRCA | wtBRCA | NA | NA | NA |
| TCGA-24-0980 | 42 | 0.221966515 | 14 | wtBRCA | wtBRCA | NA | NA | NA |
| TCGA-04-1350 | 35 | 0.32798786 | 12 | wtBRCA | wtBRCA | NA | NA | NA |
| TCGA-24-2260 | 50 | 0.429672248 | 29 | wtBRCA | wtBRCA | NA | NA | NA |
| TCGA-24-1553 | 33 | 0.489965574 | 26 | wtBRCA | wtBRCA | NA | NA | NA |
| TCGA-24-1466 | 52 | 0.265449996 | 25 | wtBRCA | wtBRCA | NA | NA | NA |
| TCGA-23-1028 | 49 | 0.42404343 | 33 | wtBRCA | wtBRCA | NA | NA | NA |
| TCGA-09-1662 | 34 | 0.31380364 | 21 | wtBRCA | wtBRCA | NA | NA | NA |
| TCGA-59-2363 | 86 | NA | NA | wtBRCA | wtBRCA | NA | NA | NA |
| TCGA-04-1337 | 65 | 0.21759332 | 10 | wtBRCA | wtBRCA | NA | NA | NA |
| TCGA-25-1315 | 59 | 0.319785324 | 24 | wtBRCA | wtBRCA | NA | NA | NA |
| TCGA-25-2396 | 32 | 0.23760573 | 16 | wtBRCA | wtBRCA | NA | NA | NA |
| TCGA-25-1627 | 31 | 0.481745012 | 24 | wtBRCA | wtBRCA | NA | NA | NA |
| TCGA-04-1361 | 69 | 0.355082194 | 22 | wtBRCA | wtBRCA | NA | NA | NA |
| TCGA-04-1362 | 76 | 0.301017795 | 32 | wtBRCA | wtBRCA | NA | NA | NA |
| TCGA-09-1665 | 96 | 0.471605894 | 34 | wtBRCA | wtBRCA | NA | NA | NA |
| TCGA-09-2044 | 98 | 0.442043 | 31 | wtBRCA | wtBRCA | NA | NA | NA |
| TCGA-10-0928 | 44 | 0.501214217 | 37 | wtBRCA | wtBRCA | NA | NA | NA |
| TCGA-13-0897 | 56 | 0.288755253 | 24 | wtBRCA | wtBRCA | NA | NA | NA |
| TCGA-13-0905 | 66 | 0.315238843 | 25 | wtBRCA | wtBRCA | NA | NA | NA |
| TCGA-13-0916 | 73 | 0.512313421 | 27 | wtBRCA | wtBRCA | NA | NA | NA |
| TCGA-13-0920 | 129 | 0.358580148 | 33 | wtBRCA | wtBRCA | NA | NA | NA |
| TCGA-13-1482 | 71 | 0.3190285 | 23 | wtBRCA | wtBRCA | NA | NA | NA |
| TCGA-13-1483 | 67 | 0.29463532 | 24 | wtBRCA | wtBRCA | NA | NA | NA |
| TCGA-13-1497 | 147 | 0.457444859 | 34 | wtBRCA | wtBRCA | NA | NA | NA |
| TCGA-13-1510 | 116 | NA | NA | wtBRCA | wtBRCA | NA | NA | NA |
| TCGA-23-1022 | 210 | 0.264684634 | 29 | wtBRCA | wtBRCA | NA | NA | NA |
| TCGA-23-1117 | 115 | 0.360446633 | 32 | wtBRCA | wtBRCA | NA | NA | NA |
| TCGA-24-1423 | 70 | 0.316659654 | 26 | wtBRCA | wtBRCA | NA | NA | NA |
| TCGA-24-1425 | 33 | 0.410208915 | 23 | wtBRCA | wtBRCA | NA | NA | NA |
| TCGA-24-1428 | 16 | 0.551273963 | 35 | wtBRCA | wtBRCA | NA | NA | NA |
| TCGA-24-1435 | 86 | 0.247229523 | 27 | wtBRCA | wtBRCA | NA | NA | NA |
| TCGA-24-1557 | 48 | 0.289089185 | 28 | wtBRCA | wtBRCA | NA | NA | NA |
| TCGA-24-1567 | 51 | 0.458842301 | 29 | wtBRCA | wtBRCA | NA | NA | NA |
| TCGA-24-1614 | 39 | 0.40348848 | 27 | wtBRCA | wtBRCA | NA | NA | NA |
| TCGA-24-2289 | 149 | 0.505695657 | 32 | wtBRCA | wtBRCA | NA | NA | NA |
| TCGA-24-2290 | 61 | 0.38920045 | 29 | wtBRCA | wtBRCA | NA | NA | NA |
| TCGA-25-1313 | 146 | 0.359556232 | 31 | wtBRCA | wtBRCA | NA | NA | NA |
| TCGA-25-1326 | 143 | NA | NA | wtBRCA | wtBRCA | NA | NA | NA |
| TCGA-25-2042 | 82 | 0.392733492 | 25 | wtBRCA | wtBRCA | NA | NA | NA |
| TCGA-30-1891 | 69 | 0.460008143 | 23 | wtBRCA | wtBRCA | NA | NA | NA |
| TCGA-36-1568 | 44 | 0.255819765 | 25 | wtBRCA | wtBRCA | NA | NA | NA |
| TCGA-04-1332 | 35 | 0.207721201 | 13 | wtBRCA | wtBRCA | NA | NA | NA |
| TCGA-04-1338 | 143 | 0.356871297 | 23 | wtBRCA | wtBRCA | NA | NA | NA |
| TCGA-04-1342 | 89 | 0.267442296 | 12 | wtBRCA | wtBRCA | NA | NA | NA |
| TCGA-04-1343 | 71 | 0.323676519 | 19 | wtBRCA | wtBRCA | NA | NA | NA |
| TCGA-04-1346 | 53 | 0.327427371 | 23 | wtBRCA | wtBRCA | NA | NA | NA |
| TCGA-04-1347 | 130 | 0.295401557 | 21 | wtBRCA | wtBRCA | NA | NA | NA |
| TCGA-04-1348 | 46 | 0.222745161 | 19 | wtBRCA | wtBRCA | NA | NA | NA |
| TCGA-04-1349 | 38 | 0.363120041 | 21 | wtBRCA | wtBRCA | NA | NA | NA |
| TCGA-04-1364 | 39 | NA | NA | wtBRCA | wtBRCA | NA | NA | NA |
| TCGA-04-1365 | 38 | 0.281770702 | 15 | wtBRCA | wtBRCA | NA | NA | NA |
| TCGA-04-1514 | 31 | 0.410294534 | 22 | wtBRCA | wtBRCA | NA | NA | NA |
| TCGA-04-1517 | 20 | 0.394639821 | 20 | wtBRCA | wtBRCA | NA | NA | NA |
| TCGA-04-1525 | 21 | 0.220344723 | 16 | wtBRCA | wtBRCA | NA | NA | NA |
| TCGA-04-1530 | 70 | 0.233030655 | 26 | wtBRCA | wtBRCA | NA | NA | NA |
| TCGA-04-1542 | 73 | 0.346531589 | 16 | wtBRCA | wtBRCA | NA | NA | NA |
| TCGA-09-0366 | 45 | NA | NA | wtBRCA | wtBRCA | NA | NA | NA |
| TCGA-09-0369 | 69 | 0.382129405 | 24 | wtBRCA | wtBRCA | NA | NA | NA |
| TCGA-09-1659 | 18 | 0.24058678 | 20 | wtBRCA | wtBRCA | NA | NA | NA |
| TCGA-09-1661 | 40 | 0.398082457 | 20 | wtBRCA | wtBRCA | NA | NA | NA |
| TCGA-09-1666 | 18 | 0.394960435 | 28 | wtBRCA | wtBRCA | NA | NA | NA |
| TCGA-09-2049 | 127 | 0.217249972 | 30 | wtBRCA | wtBRCA | NA | NA | NA |
| TCGA-09-2053 | 53 | 0.445958001 | 23 | wtBRCA | wtBRCA | NA | NA | NA |
| TCGA-10-0926 | 44 | 0.233863966 | 7 | wtBRCA | wtBRCA | NA | NA | NA |
| TCGA-10-0927 | 28 | 0.350017278 | 15 | wtBRCA | wtBRCA | NA | NA | NA |
| TCGA-10-0930 | 174 | 0.397637599 | 32 | wtBRCA | wtBRCA | NA | NA | NA |
| TCGA-10-0933 | 45 | 0.250110363 | 20 | wtBRCA | wtBRCA | NA | NA | NA |
| TCGA-10-0934 | 28 | 0.076384306 | 4 | wtBRCA | wtBRCA | NA | NA | NA |
| TCGA-10-0935 | 50 | 0.230478381 | 13 | wtBRCA | wtBRCA | NA | NA | NA |
| TCGA-10-0937 | 41 | 0.322935598 | 27 | wtBRCA | wtBRCA | NA | NA | NA |
| TCGA-10-0938 | 56 | 0.216687696 | 21 | wtBRCA | wtBRCA | NA | NA | NA |
| TCGA-13-0714 | 69 | 0.314132373 | 32 | wtBRCA | wtBRCA | NA | NA | NA |
| TCGA-13-0717 | 41 | 0.337736583 | 19 | wtBRCA | wtBRCA | NA | NA | NA |
| TCGA-13-0720 | 55 | 0.386797238 | 19 | wtBRCA | wtBRCA | NA | NA | NA |
| TCGA-13-0723 | 51 | 0.310925977 | 18 | wtBRCA | wtBRCA | NA | NA | NA |
| TCGA-13-0724 | 49 | 0.240768207 | 23 | wtBRCA | wtBRCA | NA | NA | NA |
| TCGA-13-0727 | 31 | 0.196343025 | 16 | wtBRCA | wtBRCA | NA | NA | NA |
| TCGA-13-0751 | 45 | 0.242813072 | 19 | wtBRCA | wtBRCA | NA | NA | NA |
| TCGA-13-0755 | 101 | 0.271431312 | 25 | wtBRCA | wtBRCA | NA | NA | NA |
| TCGA-13-0762 | 68 | 0.188798997 | 21 | wtBRCA | wtBRCA | NA | NA | NA |
| TCGA-13-0765 | 30 | 0.421688064 | 17 | wtBRCA | wtBRCA | NA | NA | NA |
| TCGA-13-0791 | 81 | 0.146352959 | 19 | wtBRCA | wtBRCA | NA | NA | NA |
| TCGA-13-0795 | 74 | 0.171611208 | 20 | wtBRCA | wtBRCA | NA | NA | NA |
| TCGA-13-0807 | 64 | 0.256237077 | 18 | wtBRCA | wtBRCA | NA | NA | NA |
| TCGA-13-0884 | 99 | 0.199544126 | 21 | wtBRCA | wtBRCA | NA | NA | NA |
| TCGA-13-0889 | 30 | 0.219468584 | 18 | wtBRCA | wtBRCA | NA | NA | NA |
| TCGA-13-0891 | 31 | 0.307210699 | 20 | wtBRCA | wtBRCA | NA | NA | NA |
| TCGA-13-0894 | 62 | 0.4589616 | 22 | wtBRCA | wtBRCA | NA | NA | NA |
| TCGA-13-0899 | 43 | 0.172624303 | 24 | wtBRCA | wtBRCA | NA | NA | NA |
| TCGA-13-0904 | 118 | 0.388945223 | 31 | wtBRCA | wtBRCA | NA | NA | NA |
| TCGA-13-0910 | 31 | 0.394614193 | 13 | wtBRCA | wtBRCA | NA | NA | NA |
| TCGA-13-0911 | 25 | 0.419590761 | 20 | wtBRCA | wtBRCA | NA | NA | NA |
| TCGA-13-0912 | 38 | 0.183869883 | 15 | wtBRCA | wtBRCA | NA | NA | NA |
| TCGA-13-0919 | 60 | 0.263726841 | 17 | wtBRCA | wtBRCA | NA | NA | NA |
| TCGA-13-1403 | 54 | 0.266415998 | 22 | wtBRCA | wtBRCA | NA | NA | NA |
| TCGA-13-1404 | 62 | 0.374046095 | 23 | wtBRCA | wtBRCA | NA | NA | NA |
| TCGA-13-1405 | 38 | 0.372656011 | 17 | wtBRCA | wtBRCA | NA | NA | NA |
| TCGA-13-1407 | 35 | 0.155714765 | 23 | wtBRCA | wtBRCA | NA | NA | NA |
| TCGA-13-1409 | 57 | 0.204740808 | 14 | wtBRCA | wtBRCA | NA | NA | NA |
| TCGA-13-1410 | 66 | NA | NA | wtBRCA | wtBRCA | NA | NA | NA |
| TCGA-13-1411 | 54 | NA | NA | wtBRCA | wtBRCA | NA | NA | NA |
| TCGA-13-1412 | 39 | NA | NA | wtBRCA | wtBRCA | NA | NA | NA |
| TCGA-13-1484 | 45 | 0.363908919 | 11 | wtBRCA | wtBRCA | NA | NA | NA |
| TCGA-13-1487 | 45 | 0.288943112 | 21 | wtBRCA | wtBRCA | NA | NA | NA |
| TCGA-13-1488 | 130 | 0.16825819 | 23 | wtBRCA | wtBRCA | NA | NA | NA |
| TCGA-13-1491 | 47 | 0.543160595 | 19 | wtBRCA | wtBRCA | NA | NA | NA |
| TCGA-13-1492 | 45 | 0.365492473 | 13 | wtBRCA | wtBRCA | NA | NA | NA |
| TCGA-13-1495 | 44 | 0.248362158 | 24 | wtBRCA | wtBRCA | NA | NA | NA |
| TCGA-13-1504 | 38 | 0.254438518 | 22 | wtBRCA | wtBRCA | NA | NA | NA |
| TCGA-13-1505 | 65 | NA | NA | wtBRCA | wtBRCA | NA | NA | NA |
| TCGA-13-1506 | 30 | NA | NA | wtBRCA | wtBRCA | NA | NA | NA |
| TCGA-13-1507 | 95 | NA | NA | wtBRCA | wtBRCA | NA | NA | NA |
| TCGA-13-1509 | 99 | NA | NA | wtBRCA | wtBRCA | NA | NA | NA |
| TCGA-13-2060 | 48 | 0.284641103 | 24 | wtBRCA | wtBRCA | NA | NA | NA |
| TCGA-20-0987 | 27 | 0.23427386 | 18 | wtBRCA | wtBRCA | NA | NA | NA |
| TCGA-20-0991 | 85 | 0.156413263 | 15 | wtBRCA | wtBRCA | NA | NA | NA |
| TCGA-23-1021 | 95 | 0.514990827 | 25 | wtBRCA | wtBRCA | NA | NA | NA |
| TCGA-23-1023 | 41 | 0.231793929 | 16 | wtBRCA | wtBRCA | NA | NA | NA |
| TCGA-23-1024 | 41 | 0.096359427 | 18 | wtBRCA | wtBRCA | NA | NA | NA |
| TCGA-23-1032 | 100 | 0.205605131 | 23 | wtBRCA | wtBRCA | NA | NA | NA |
| TCGA-23-1116 | 62 | NA | NA | wtBRCA | wtBRCA | NA | NA | NA |
| TCGA-23-1124 | 163 | 0.328242727 | 35 | wtBRCA | wtBRCA | NA | NA | NA |
| TCGA-23-2072 | 57 | 0.284235089 | 18 | wtBRCA | wtBRCA | NA | NA | NA |
| TCGA-24-0966 | 40 | 0.153105559 | 15 | wtBRCA | wtBRCA | NA | NA | NA |
| TCGA-24-0968 | 22 | 0.21283391 | 22 | wtBRCA | wtBRCA | NA | NA | NA |
| TCGA-24-0970 | 28 | 0.462417483 | 23 | wtBRCA | wtBRCA | NA | NA | NA |
| TCGA-24-0979 | 98 | 0.18808334 | 20 | wtBRCA | wtBRCA | NA | NA | NA |
| TCGA-24-0982 | 56 | 0.348719182 | 20 | wtBRCA | wtBRCA | NA | NA | NA |
| TCGA-24-1105 | 20 | 0.245223999 | 17 | wtBRCA | wtBRCA | NA | NA | NA |
| TCGA-24-1413 | 43 | 0.339773553 | 20 | wtBRCA | wtBRCA | NA | NA | NA |
| TCGA-24-1416 | 17 | 0.188316899 | 9 | wtBRCA | wtBRCA | NA | NA | NA |
| TCGA-24-1418 | 47 | 0.297964272 | 16 | wtBRCA | wtBRCA | NA | NA | NA |
| TCGA-24-1419 | 39 | 0.263572631 | 18 | wtBRCA | wtBRCA | NA | NA | NA |
| TCGA-24-1424 | 54 | 0.150461825 | 30 | wtBRCA | wtBRCA | NA | NA | NA |
| TCGA-24-1426 | 33 | NA | NA | wtBRCA | wtBRCA | NA | NA | NA |
| TCGA-24-1427 | 61 | NA | NA | wtBRCA | wtBRCA | NA | NA | NA |
| TCGA-24-1431 | 56 | 0.209317803 | 21 | wtBRCA | wtBRCA | NA | NA | NA |
| TCGA-24-1434 | 52 | 0.264662803 | 16 | wtBRCA | wtBRCA | NA | NA | NA |
| TCGA-24-1436 | 50 | 0.359079446 | 16 | wtBRCA | wtBRCA | NA | NA | NA |
| TCGA-24-1464 | 64 | 0.302346607 | 12 | wtBRCA | wtBRCA | NA | NA | NA |
| TCGA-24-1469 | 165 | 0.451134641 | 27 | wtBRCA | wtBRCA | NA | NA | NA |
| TCGA-24-1471 | 28 | NA | NA | wtBRCA | wtBRCA | NA | NA | NA |
| TCGA-24-1474 | 60 | 0.154262888 | 22 | wtBRCA | wtBRCA | NA | NA | NA |
| TCGA-24-1544 | 26 | 0.180183419 | 16 | wtBRCA | wtBRCA | NA | NA | NA |
| TCGA-24-1545 | 20 | 0.407541385 | 13 | wtBRCA | wtBRCA | NA | NA | NA |
| TCGA-24-1548 | 31 | 0.474010671 | 20 | wtBRCA | wtBRCA | NA | NA | NA |
| TCGA-24-1549 | 44 | 0.249003667 | 17 | wtBRCA | wtBRCA | NA | NA | NA |
| TCGA-24-1551 | 41 | 0.220921151 | 17 | wtBRCA | wtBRCA | NA | NA | NA |
| TCGA-24-1552 | 41 | 0.221841966 | 20 | wtBRCA | wtBRCA | NA | NA | NA |
| TCGA-24-1556 | 55 | 0.289117754 | 26 | wtBRCA | wtBRCA | NA | NA | NA |
| TCGA-24-1558 | 26 | 0.220738001 | 20 | wtBRCA | wtBRCA | NA | NA | NA |
| TCGA-24-1560 | 29 | 0.095495374 | 1 | wtBRCA | wtBRCA | NA | NA | NA |
| TCGA-24-1563 | 74 | 0.274182765 | 21 | wtBRCA | wtBRCA | NA | NA | NA |
| TCGA-24-1564 | 46 | 0.299093939 | 13 | wtBRCA | wtBRCA | NA | NA | NA |
| TCGA-24-1565 | 34 | 0.117729131 | 7 | wtBRCA | wtBRCA | NA | NA | NA |
| TCGA-24-1603 | 28 | 0.360079377 | 17 | wtBRCA | wtBRCA | NA | NA | NA |
| TCGA-24-1604 | 65 | 0.229462898 | 25 | wtBRCA | wtBRCA | NA | NA | NA |
| TCGA-24-1616 | 64 | 0.176110853 | 13 | wtBRCA | wtBRCA | NA | NA | NA |
| TCGA-24-2019 | 39 | NA | NA | wtBRCA | wtBRCA | NA | NA | NA |
| TCGA-24-2030 | 58 | 0.264164288 | 25 | wtBRCA | wtBRCA | NA | NA | NA |
| TCGA-24-2038 | 11 | NA | NA | wtBRCA | wtBRCA | NA | NA | NA |
| TCGA-24-2254 | 54 | 0.455709378 | 26 | wtBRCA | wtBRCA | NA | NA | NA |
| TCGA-24-2261 | 43 | 0.417757491 | 22 | wtBRCA | wtBRCA | NA | NA | NA |
| TCGA-24-2262 | 78 | 0.301785455 | 17 | wtBRCA | wtBRCA | NA | NA | NA |
| TCGA-24-2271 | 32 | 0.414426491 | 11 | wtBRCA | wtBRCA | NA | NA | NA |
| TCGA-24-2281 | 55 | 0.146939958 | 15 | wtBRCA | wtBRCA | NA | NA | NA |
| TCGA-25-1316 | 21 | 0.027126665 | 2 | wtBRCA | wtBRCA | NA | NA | NA |
| TCGA-25-1317 | 44 | 0.351848131 | 10 | wtBRCA | wtBRCA | NA | NA | NA |
| TCGA-25-1319 | 58 | 0.063097256 | 12 | wtBRCA | wtBRCA | NA | NA | NA |
| TCGA-25-1320 | 56 | NA | NA | wtBRCA | wtBRCA | NA | NA | NA |
| TCGA-25-1321 | 40 | 0.469399438 | 30 | wtBRCA | wtBRCA | NA | NA | NA |
| TCGA-25-1322 | 48 | 0.317101271 | 17 | wtBRCA | wtBRCA | NA | NA | NA |
| TCGA-25-1324 | 42 | NA | NA | wtBRCA | wtBRCA | NA | NA | NA |
| TCGA-25-1328 | 13 | NA | NA | wtBRCA | wtBRCA | NA | NA | NA |
| TCGA-25-1329 | 39 | NA | NA | wtBRCA | wtBRCA | NA | NA | NA |
| TCGA-25-1623 | 11 | 0.272584565 | 14 | wtBRCA | wtBRCA | NA | NA | NA |
| TCGA-25-1626 | 9 | 0.527964781 | 15 | wtBRCA | wtBRCA | NA | NA | NA |
| TCGA-25-1628 | 24 | 0.206911946 | 12 | wtBRCA | wtBRCA | NA | NA | NA |
| TCGA-25-1631 | 27 | 0.230353511 | 13 | wtBRCA | wtBRCA | NA | NA | NA |
| TCGA-25-1633 | 14 | 0.368410355 | 16 | wtBRCA | wtBRCA | NA | NA | NA |
| TCGA-25-1635 | 18 | 0.319404136 | 17 | wtBRCA | wtBRCA | NA | NA | NA |
| TCGA-25-2393 | 55 | 0.162103689 | 21 | wtBRCA | wtBRCA | NA | NA | NA |
| TCGA-25-2398 | 54 | 0.156433644 | 25 | wtBRCA | wtBRCA | NA | NA | NA |
| TCGA-25-2400 | 78 | 0.249024155 | 17 | wtBRCA | wtBRCA | NA | NA | NA |
| TCGA-25-2408 | 17 | 0.105999701 | 4 | wtBRCA | wtBRCA | NA | NA | NA |
| TCGA-25-2409 | 28 | 0.204794851 | 14 | wtBRCA | wtBRCA | NA | NA | NA |
| TCGA-30-1853 | 46 | 0.29625011 | 20 | wtBRCA | wtBRCA | NA | NA | NA |
| TCGA-30-1862 | 38 | 0.287929574 | 19 | wtBRCA | wtBRCA | NA | NA | NA |
| TCGA-31-1950 | 48 | 0.364031743 | 21 | wtBRCA | wtBRCA | NA | NA | NA |
| TCGA-31-1953 | 38 | 0.19286638 | 20 | wtBRCA | wtBRCA | NA | NA | NA |
| TCGA-31-1959 | 54 | NA | NA | wtBRCA | wtBRCA | NA | NA | NA |
| TCGA-36-1569 | 10 | 0.372741221 | 23 | wtBRCA | wtBRCA | NA | NA | NA |
| TCGA-36-1570 | 30 | 0.240491333 | 21 | wtBRCA | wtBRCA | NA | NA | NA |
| TCGA-36-1571 | 13 | 0.236103881 | 18 | wtBRCA | wtBRCA | NA | NA | NA |
| TCGA-36-1574 | 20 | 0.360391213 | 24 | wtBRCA | wtBRCA | NA | NA | NA |
| TCGA-36-1575 | 34 | 0.259453942 | 15 | wtBRCA | wtBRCA | NA | NA | NA |
| TCGA-36-1576 | 17 | 0.113016942 | 12 | wtBRCA | wtBRCA | NA | NA | NA |
| TCGA-36-1577 | 62 | 0.400543558 | 29 | wtBRCA | wtBRCA | NA | NA | NA |
| TCGA-36-1578 | 57 | NA | NA | wtBRCA | wtBRCA | NA | NA | NA |
| TCGA-36-1580 | 14 | 0.303294085 | 25 | wtBRCA | wtBRCA | NA | NA | NA |
| TCGA-57-1583 | 10 | 0.215603939 | 20 | wtBRCA | wtBRCA | NA | NA | NA |
| TCGA-57-1993 | 57 | 0.049678172 | 16 | wtBRCA | wtBRCA | NA | NA | NA |
| TCGA-59-2350 | 32 | 0.422125019 | 10 | wtBRCA | wtBRCA | NA | NA | NA |
| TCGA-59-2352 | 85 | 0.245532101 | 21 | wtBRCA | wtBRCA | NA | NA | NA |
| TCGA-59-2355 | 27 | 0.309882187 | 13 | wtBRCA | wtBRCA | NA | NA | NA |
| TCGA-61-1728 | 35 | 0.14667758 | 19 | wtBRCA | wtBRCA | NA | NA | NA |
| TCGA-61-1736 | 41 | 0.322972716 | 13 | wtBRCA | wtBRCA | NA | NA | NA |
| TCGA-61-1919 | 39 | NA | NA | wtBRCA | wtBRCA | NA | NA | NA |
| TCGA-61-1995 | 22 | NA | NA | wtBRCA | wtBRCA | NA | NA | NA |
| TCGA-61-1998 | 106 | NA | NA | wtBRCA | wtBRCA | NA | NA | NA |
| TCGA-61-2000 | 46 | 0.258582816 | 19 | wtBRCA | wtBRCA | NA | NA | NA |
| TCGA-61-2002 | 44 | 0.377985376 | 28 | wtBRCA | wtBRCA | NA | NA | NA |
| TCGA-61-2003 | 45 | NA | NA | wtBRCA | wtBRCA | NA | NA | NA |
| TCGA-61-2009 | 64 | 0.25808357 | 17 | wtBRCA | wtBRCA | NA | NA | NA |
| TCGA-61-2012 | 112 | 0.322370988 | 23 | wtBRCA | wtBRCA | NA | NA | NA |
| TCGA-61-2016 | 21 | 0.39587497 | 19 | wtBRCA | wtBRCA | NA | NA | NA |
| TCGA-61-2088 | 19 | NA | NA | wtBRCA | wtBRCA | NA | NA | NA |
| TCGA-61-2092 | 33 | 0.022379319 | 4 | wtBRCA | wtBRCA | NA | NA | NA |
| TCGA-61-2094 | 57 | 0.286411222 | 16 | wtBRCA | wtBRCA | NA | NA | NA |
| TCGA-61-2097 | 52 | 0.424029098 | 23 | wtBRCA | wtBRCA | NA | NA | NA |
| TCGA-61-2101 | 39 | 0.368887803 | 16 | wtBRCA | wtBRCA | NA | NA | NA |
| TCGA-61-2102 | 63 | 0.319640628 | 22 | wtBRCA | wtBRCA | NA | NA | NA |
| TCGA-61-2104 | 56 | 0.319122043 | 29 | wtBRCA | wtBRCA | NA | NA | NA |
| TCGA-61-2110 | 45 | 0.294406689 | 8 | wtBRCA | wtBRCA | NA | NA | NA |
| TCGA-61-2111 | 45 | NA | NA | wtBRCA | wtBRCA | NA | NA | NA |
| TCGA-61-2113 | 80 | NA | NA | wtBRCA | wtBRCA | NA | NA | NA |

| | mBRCA2 | BRCA1 | RAD51C | | |
|---|---|---|---|---|---|
| Patient ID | LOH status | methylation | methylation | Jewish origin | Race |
| TCGA-04-1331 | Het loss | No | No | No | WHITE |
| TCGA-04-1336 | Het loss | No | No | No | WHITE |
| TCGA-04-1356 | NA | No | No | No | HISPANIC OR LATINO |
| TCGA-04-1357 | NA | No | No | No | [Not Available] |
| TCGA-04-1367 | Het loss | No | No | No | WHITE |
| TCGA-09-1669 | NA | No | No | No | WHITE |
| TCGA-09-2045 | NA | No | No | No | ASIAN |
| TCGA-09-2050 | Het loss | No | No | No | WHITE |
| TCGA-09-2051 | NA | No | No | ASHKENAZI | WHITE |
| TCGA-10-0931 | NA | No | No | No | WHITE |
| TCGA-13-0726 | Het loss | No | No | No | WHITE |
| TCGA-13-0730 | NA | No | No | No | WHITE |
| TCGA-13-0761 | NA | No | No | No | WHITE |
| TCGA-13-0792 | Diploid | No | No | No | WHITE |
| TCGA-13-0793 | Het loss | No | No | No | WHITE |
| TCGA-13-0804 | NA | No | No | No | WHITE |
| TCGA-13-0883 | NA | No | No | ASHKENAZI | WHITE |
| TCGA-13-0885 | Het loss | No | No | No | WHITE |
| TCGA-13-0886 | Het loss | No | No | No | WHITE |
| TCGA-13-0887 | NA | No | No | ASHKENAZI | WHITE |
| TCGA-13-0890 | Het loss | No | No | ASHKENAZI | WHITE |
| TCGA-13-0893 | NA | No | No | No | BLACK OR AFRICAN |
| | | | | | AMERICAN |
| TCGA-13-0900 | Het loss | No | No | No | WHITE |
| TCGA-13-0903 | NA | No | No | No | WHITE |
| TCGA-13-0913 | Het loss | No | No | No | WHITE |
| TCGA-13-1408 | NA | No | No | ASHKENAZI | WHITE |
| TCGA-13-1481 | Diploid | No | No | No | WHITE |
| TCGA-13-1489 | NA | No | No | No | WHITE |
| TCGA-13-1494 | NA | No | No | No | WHITE |
| TCGA-13-1498 | Diploid | No | No | ASHKENAZI | WHITE |
| TCGA-13-1499 | Het loss | No | No | ASHKENAZI | WHITE |
| TCGA-13-1512 | Diploid | No | No | No | WHITE |
| TCGA-23-1026 | Diploid | No | No | No | WHITE |
| TCGA-23-1027 | NA | No | No | No | WHITE |
| TCGA-23-1030 | Diploid | No | No | No | WHITE |
| TCGA-23-1118 | NA | No | No | No | WHITE |
| TCGA-23-1120 | Het loss | No | No | No | WHITE |
| TCGA-23-1122 | NA | No | No | No | WHITE |
| TCGA-23-2077 | NA | No | No | No | WHITE |
| TCGA-23-2078 | NA | No | No | ASHKENAZI | WHITE |
| TCGA-23-2079 | NA | No | No | ASHKENAZI | WHITE |
| TCGA-23-2081 | NA | No | No | ASHKENAZI | WHITE |
| TCGA-24-0975 | Het loss | No | No | No | WHITE |
| TCGA-24-1103 | Het loss | No | No | No | BLACK OR AFRICAN |
| | | | | | AMERICAN |
| TCGA-24-1417 | Het loss | No | No | No | WHITE |
| TCGA-24-1463 | Diploid | No | No | No | WHITE |
| TCGA-24-1470 | NA | No | No | No | WHITE |
| TCGA-24-1555 | Het loss | No | No | No | WHITE |
| TCGA-24-1562 | Diploid | No | No | No | WHITE |
| TCGA-24-2024 | Het loss | No | No | No | BLACK OR AFRICAN |
| | | | | | AMERICAN |
| TCGA-24-2035 | NA | No | No | No | WHITE |
| TCGA-24-2280 | Het loss | No | No | No | WHITE |
| TCGA-24-2288 | Het loss | No | No | No | WHITE |
| TCGA-24-2293 | Diploid | No | No | No | WHITE |
| TCGA-24-2298 | NA | No | No | No | WHITE |
| TCGA-25-1318 | Het loss | No | No | No | WHITE |
| TCGA-25-1625 | NA | No | No | No | WHITE |
| TCGA-25-1630 | NA | No | No | No | WHITE |
| TCGA-25-1632 | NA | No | No | No | WHITE |
| TCGA-25-1634 | Het loss | No | No | No | WHITE |
| TCGA-25-2392 | NA | No | No | No | WHITE |
| TCGA-25-2401 | NA | No | No | No | WHITE |
| TCGA-25-2404 | Het loss | No | No | No | AMERICAN INDIAN |
| | | | | | OR ALASKA NATIVE |
| TCGA-29-2427 | NA | No | No | No | WHITE |
| TCGA-57-1582 | NA | No | No | No | WHITE |
| TCGA-57-1584 | Het loss | No | No | No | WHITE |
| TCGA-59-2348 | NA | No | No | No | WHITE |
| TCGA-59-2351 | Het loss | No | No | No | WHITE |
| TCGA-61-2008 | NA | No | No | No | ASIAN |
| TCGA-61-2109 | NA | No | No | No | WHITE |
| TCGA-13-1501 | NA | Yes | No | No | WHITE |
| TCGA-13-0800 | NA | No | No | No | WHITE |
| TCGA-13-0760 | NA | Yes | No | No | WHITE |
| TCGA-13-0906 | NA | No | No | No | WHITE |
| TCGA-61-2095 | NA | No | No | No | WHITE |
| TCGA-23-1123 | NA | No | No | No | WHITE |
| TCGA-20-0990 | NA | No | No | No | WHITE |
| TCGA-13-0923 | NA | No | No | No | WHITE |
| TCGA-13-1496 | NA | No | No | No | WHITE |
| TCGA-24-2267 | NA | No | No | No | WHITE |
| TCGA-09-2056 | NA | No | No | No | HISPANIC OR LATINO |
| TCGA-13-1477 | NA | No | No | No | WHITE |
| TCGA-24-1422 | NA | No | Yes | No | BLACK OR AFRICAN |
| | | | | | AMERICAN |
| TCGA-23-1110 | NA | No | Yes | No | HISPANIC OR LATINO |
| TCGA-23-1031 | NA | No | Yes | ASHKENAZI | WHITE |
| TCGA-13-0924 | NA | No | Yes | ASHKENAZI | WHITE |
| TCGA-24-1104 | NA | No | Yes | No | WHITE |
| TCGA-25-2391 | NA | No | Yes | No | WHITE |
| TCGA-59-2354 | NA | No | Yes | No | WHITE |
| TCGA-25-2399 | NA | No | No | No | WHITE |
| TCGA-24-0980 | NA | No | No | No | WHITE |
| TCGA-04-1350 | NA | No | No | No | WHITE |
| TCGA-24-2260 | NA | No | No | No | WHITE |
| TCGA-24-1553 | NA | No | No | No | WHITE |
| TCGA-24-1466 | NA | No | No | No | WHITE |
| TCGA-23-1028 | NA | No | No | No | HISPANIC OR LATINO |
| TCGA-09-1662 | NA | No | Yes | No | WHITE |
| TCGA-59-2363 | NA | No | No | No | ASIAN |
| TCGA-04-1337 | NA | No | No | No | WHITE |
| TCGA-25-1315 | NA | No | No | No | WHITE |
| TCGA-25-2396 | NA | No | No | No | WHITE |
| TCGA-25-1627 | NA | No | No | No | WHITE |
| TCGA-04-1361 | NA | Yes | No | No | WHITE |
| TCGA-04-1362 | NA | Yes | No | No | WHITE |
| TCGA-09-1665 | NA | Yes | No | No | WHITE |
| TCGA-09-2044 | NA | Yes | No | No | ASIAN |
| TCGA-10-0928 | NA | Yes | No | No | WHITE |
| TCGA-13-0897 | NA | Yes | No | ASHKENAZI | WHITE |
| TCGA-13-0905 | NA | Yes | No | No | WHITE |
| TCGA-13-0916 | NA | Yes | No | No | WHITE |
| TCGA-13-0920 | NA | Yes | No | No | WHITE |
| TCGA-13-1482 | NA | Yes | No | No | WHITE |
| TCGA-13-1483 | NA | Yes | No | No | WHITE |
| TCGA-13-1497 | NA | Yes | No | No | WHITE |
| TCGA-13-1510 | NA | Yes | No | No | WHITE |
| TCGA-23-1022 | NA | Yes | No | No | WHITE |
| TCGA-23-1117 | NA | Yes | No | No | WHITE |
| TCGA-24-1423 | NA | Yes | No | No | WHITE |
| TCGA-24-1425 | NA | Yes | No | No | WHITE |
| TCGA-24-1428 | NA | Yes | No | No | WHITE |
| TCGA-24-1435 | NA | Yes | No | No | WHITE |
| TCGA-24-1557 | NA | Yes | No | No | WHITE |
| TCGA-24-1567 | NA | Yes | No | No | WHITE |
| TCGA-24-1614 | NA | Yes | No | No | WHITE |
| TCGA-24-2289 | NA | Yes | No | No | WHITE |
| TCGA-24-2290 | NA | Yes | No | No | WHITE |
| TCGA-25-1313 | NA | Yes | No | No | WHITE |
| TCGA-25-1326 | NA | Yes | No | No | WHITE |
| TCGA-25-2042 | NA | Yes | No | No | AMERICAN INDIAN |
| | | | | | OR ALASKA NATIVE |
| TCGA-30-1891 | NA | Yes | No | No | WHITE |
| TCGA-36-1568 | NA | Yes | No | No | [Not Available] |
| TCGA-04-1332 | NA | No | No | No | WHITE |
| TCGA-04-1338 | NA | No | No | No | WHITE |
| TCGA-04-1342 | NA | No | No | No | WHITE |
| TCGA-04-1343 | NA | No | No | No | WHITE |
| TCGA-04-1346 | NA | No | No | No | WHITE |
| TCGA-04-1347 | NA | No | No | No | WHITE |
| TCGA-04-1348 | NA | No | No | No | HISPANIC OR LATINO |
| TCGA-04-1349 | NA | No | No | No | WHITE |
| TCGA-04-1364 | NA | No | No | No | WHITE |
| TCGA-04-1365 | NA | No | No | No | WHITE |
| TCGA-04-1514 | NA | No | No | No | WHITE |
| TCGA-04-1517 | NA | No | No | No | WHITE |
| TCGA-04-1525 | NA | No | No | No | WHITE |
| TCGA-04-1530 | NA | No | No | No | WHITE |
| TCGA-04-1542 | NA | No | No | No | WHITE |
| TCGA-09-0366 | NA | No | No | No | WHITE |
| TCGA-09-0369 | NA | No | No | No | WHITE |
| TCGA-09-1659 | NA | No | No | No | WHITE |
| TCGA-09-1661 | NA | No | No | No | WHITE |
| TCGA-09-1666 | NA | No | No | No | WHITE |
| TCGA-09-2049 | NA | No | No | No | WHITE |
| TCGA-09-2053 | NA | No | No | No | WHITE |
| TCGA-10-0926 | NA | No | No | No | WHITE |
| TCGA-10-0927 | NA | No | No | No | HISPANIC OR LATINO |
| TCGA-10-0930 | NA | No | No | No | WHITE |
| TCGA-10-0933 | NA | No | No | No | WHITE |
| TCGA-10-0934 | NA | No | No | No | WHITE |
| TCGA-10-0935 | NA | No | No | No | WHITE |
| TCGA-10-0937 | NA | No | No | No | WHITE |
| TCGA-10-0938 | NA | No | No | No | WHITE |
| TCGA-13-0714 | NA | No | No | No | WHITE |
| TCGA-13-0717 | NA | No | No | No | WHITE |
| TCGA-13-0720 | NA | No | No | No | WHITE |
| TCGA-13-0723 | NA | No | No | No | WHITE |
| TCGA-13-0724 | NA | No | No | No | HISPANIC OR LATINO |
| TCGA-13-0727 | NA | No | No | No | WHITE |
| TCGA-13-0751 | NA | No | No | No | WHITE |
| TCGA-13-0755 | NA | No | No | No | WHITE |
| TCGA-13-0762 | NA | No | No | ASHKENAZI | WHITE |
| TCGA-13-0765 | NA | No | No | No | WHITE |
| TCGA-13-0791 | NA | No | No | No | WHITE |
| TCGA-13-0795 | NA | No | No | No | WHITE |
| TCGA-13-0807 | NA | No | No | No | WHITE |
| TCGA-13-0884 | NA | No | No | No | WHITE |
| TCGA-13-0889 | NA | No | No | No | WHITE |
| TCGA-13-0891 | NA | No | No | ASHKENAZI | WHITE |
| TCGA-13-0894 | NA | No | No | No | WHITE |
| TCGA-13-0899 | NA | No | No | No | WHITE |
| TCGA-13-0904 | NA | No | No | No | WHITE |
| TCGA-13-0910 | NA | No | No | No | WHITE |
| TCGA-13-0911 | NA | No | No | No | WHITE |
| TCGA-13-0912 | NA | No | No | ASHKENAZI | WHITE |
| TCGA-13-0919 | NA | No | No | No | WHITE |
| TCGA-13-1403 | NA | No | No | No | WHITE |
| TCGA-13-1404 | NA | No | No | No | WHITE |
| TCGA-13-1405 | NA | No | No | No | WHITE |
| TCGA-13-1407 | NA | No | No | No | WHITE |
| TCGA-13-1409 | NA | No | No | No | WHITE |
| TCGA-13-1410 | NA | No | No | No | WHITE |
| TCGA-13-1411 | NA | No | No | No | WHITE |
| TCGA-13-1412 | NA | No | No | No | WHITE |
| TCGA-13-1484 | NA | No | No | No | WHITE |
| TCGA-13-1487 | NA | No | No | No | WHITE |
| TCGA-13-1488 | NA | No | No | No | WHITE |
| TCGA-13-1491 | NA | No | No | No | ASIAN |
| TCGA-13-1492 | NA | No | No | No | WHITE |
| TCGA-13-1495 | NA | No | No | No | WHITE |
| TCGA-13-1504 | NA | No | No | No | WHITE |
| TCGA-13-1505 | NA | No | No | No | WHITE |
| TCGA-13-1506 | NA | No | No | No | WHITE |
| TCGA-13-1507 | NA | No | No | No | WHITE |
| TCGA-13-1509 | NA | No | No | ASHKENAZI | WHITE |
| TCGA-13-2060 | NA | No | No | No | WHITE |
| TCGA-20-0987 | NA | No | No | No | WHITE |
| TCGA-20-0991 | NA | No | No | No | WHITE |
| TCGA-23-1021 | NA | No | No | No | WHITE |
| TCGA-23-1023 | NA | No | No | No | WHITE |
| TCGA-23-1024 | NA | No | No | ASHKENAZI | WHITE |
| TCGA-23-1032 | NA | No | No | No | BLACK OR AFRICAN |
| | | | | | AMERICAN |
| TCGA-23-1116 | NA | No | No | ASHKENAZI | WHITE |
| TCGA-23-1124 | NA | No | No | No | WHITE |
| TCGA-23-2072 | NA | No | No | ASHKENAZI | WHITE |
| TCGA-24-0966 | NA | No | No | No | BLACK OR AFRICAN |
| | | | | | AMERICAN |
| TCGA-24-0968 | NA | No | No | No | WHITE |
| TCGA-24-0970 | NA | No | No | No | WHITE |
| TCGA-24-0979 | NA | No | No | No | WHITE |
| TCGA-24-0982 | NA | No | No | No | WHITE |
| TCGA-24-1105 | NA | No | No | No | WHITE |
| TCGA-24-1413 | NA | No | No | No | WHITE |
| TCGA-24-1416 | NA | No | No | No | WHITE |
| TCGA-24-1418 | NA | No | No | No | WHITE |
| TCGA-24-1419 | NA | No | No | No | WHITE |
| TCGA-24-1424 | NA | No | No | No | WHITE |
| TCGA-24-1426 | NA | No | No | No | WHITE |
| TCGA-24-1427 | NA | No | No | No | [Not Available] |
| TCGA-24-1431 | NA | No | No | No | WHITE |
| TCGA-24-1434 | NA | No | No | No | WHITE |
| TCGA-24-1436 | NA | No | No | No | WHITE |
| TCGA-24-1464 | NA | No | No | No | WHITE |
| TCGA-24-1469 | NA | No | No | No | WHITE |
| TCGA-24-1471 | NA | No | No | No | WHITE |
| TCGA-24-1474 | NA | No | No | No | BLACK OR AFRICAN |
| | | | | | AMERICAN |
| TCGA-24-1544 | NA | No | No | No | BLACK OR AFRICAN |
| | | | | | AMERICAN |
| TCGA-24-1545 | NA | No | No | No | WHITE |
| TCGA-24-1548 | NA | No | No | No | WHITE |
| TCGA-24-1549 | NA | No | No | No | WHITE |
| TCGA-24-1551 | NA | No | No | No | WHITE |
| TCGA-24-1552 | NA | No | No | No | WHITE |
| TCGA-24-1556 | NA | No | No | No | WHITE |
| TCGA-24-1558 | NA | No | No | No | WHITE |
| TCGA-24-1560 | NA | No | No | No | WHITE |
| TCGA-24-1563 | NA | No | No | No | BLACK OR AFRICAN |
| | | | | | AMERICAN |
| TCGA-24-1564 | NA | No | No | No | WHITE |
| TCGA-24-1565 | NA | No | No | No | WHITE |
| TCGA-24-1603 | NA | No | No | No | WHITE |
| TCGA-24-1604 | NA | No | No | No | WHITE |
| TCGA-24-1616 | NA | No | No | No | WHITE |
| TCGA-24-2019 | NA | No | No | No | WHITE |
| TCGA-24-2030 | NA | No | No | No | WHITE |
| TCGA-24-2038 | NA | No | No | No | WHITE |
| TCGA-24-2254 | NA | No | No | No | WHITE |
| TCGA-24-2261 | NA | No | No | No | WHITE |
| TCGA-24-2262 | NA | No | No | No | WHITE |
| TCGA-24-2271 | NA | No | No | No | ASIAN |
| TCGA-24-2281 | NA | No | No | No | WHITE |
| TCGA-25-1316 | NA | No | No | No | WHITE |
| TCGA-25-1317 | NA | No | No | No | WHITE |
| TCGA-25-1319 | NA | No | No | No | WHITE |
| TCGA-25-1320 | NA | No | No | No | WHITE |
| TCGA-25-1321 | NA | No | No | No | WHITE |
| TCGA-25-1322 | NA | No | No | No | WHITE |
| TCGA-25-1324 | NA | No | No | No | WHITE |
| TCGA-25-1328 | NA | No | No | No | WHITE |
| TCGA-25-1329 | NA | No | No | No | WHITE |
| TCGA-25-1623 | NA | No | No | No | WHITE |
| TCGA-25-1626 | NA | No | No | No | WHITE |
| TCGA-25-1628 | NA | No | No | No | WHITE |
| TCGA-25-1631 | NA | No | No | No | WHITE |
| TCGA-25-1633 | NA | No | No | No | WHITE |
| TCGA-25-1635 | NA | No | No | No | WHITE |
| TCGA-25-2393 | NA | No | No | No | WHITE |
| TCGA-25-2398 | NA | No | No | No | WHITE |
| TCGA-25-2400 | NA | No | No | No | WHITE |
| TCGA-25-2408 | NA | No | No | No | WHITE |
| TCGA-25-2409 | NA | No | No | No | WHITE |
| TCGA-30-1853 | NA | No | No | No | WHITE |
| TCGA-30-1862 | NA | No | No | No | WHITE |
| TCGA-31-1950 | NA | No | No | No | WHITE |
| TCGA-31-1953 | NA | No | No | No | ASIAN |
| TCGA-31-1959 | NA | No | No | No | WHITE |
| TCGA-36-1569 | NA | No | No | No | WHITE |
| TCGA-36-1570 | NA | No | No | No | WHITE |
| TCGA-36-1571 | NA | No | No | No | WHITE |
| TCGA-36-1574 | NA | No | No | No | ASIAN |
| TCGA-36-1575 | NA | No | No | No | [Not Available] |
| TCGA-36-1576 | NA | No | No | No | [Not Available] |
| TCGA-36-1577 | NA | No | No | No | ASIAN |
| TCGA-36-1578 | NA | No | No | No | ASIAN |
| TCGA-36-1580 | NA | No | No | No | [Not Available] |
| TCGA-57-1583 | NA | No | No | No | BLACK OR AFRICAN |
| | | | | | AMERICAN |
| TCGA-57-1993 | NA | No | No | No | WHITE |
| TCGA-59-2350 | NA | No | No | No | [Not Available] |
| TCGA-59-2352 | NA | No | No | No | WHITE |
| TCGA-59-2355 | NA | No | No | No | WHITE |
| TCGA-61-1728 | NA | No | No | No | WHITE |
| TCGA-61-1736 | NA | No | No | No | WHITE |
| TCGA-61-1919 | NA | No | No | No | WHITE |
| TCGA-61-1995 | NA | No | No | No | WHITE |
| TCGA-61-1998 | NA | No | No | No | WHITE |
| TCGA-61-2000 | NA | No | No | No | WHITE |
| TCGA-61-2002 | NA | No | No | No | WHITE |
| TCGA-61-2003 | NA | No | No | No | WHITE |
| TCGA-61-2009 | NA | No | No | No | WHITE |
| TCGA-61-2012 | NA | No | No | No | WHITE |
| TCGA-61-2016 | NA | No | No | No | WHITE |
| TCGA-61-2088 | NA | No | No | No | WHITE |
| TCGA-61-2092 | NA | No | No | No | WHITE |
| TCGA-61-2094 | NA | No | No | No | WHITE |
| TCGA-61-2097 | NA | No | No | No | WHITE |
| TCGA-61-2101 | NA | No | No | No | WHITE |
| TCGA-61-2102 | NA | No | No | No | WHITE |
| TCGA-61-2104 | NA | No | No | No | WHITE |
| TCGA-61-2110 | NA | No | No | No | WHITE |
| TCGA-61-2111 | NA | No | No | No | WHITE |
| TCGA-61-2113 | NA | No | No | No | WHITE |

Footnotes: for pathological and clinical data, see Supplementary Table 2010-09-11380C-Table_S1.2 of reference 9 at website: (http://www.nature.com/nature/journal/v474/n7353/full/nature10166.html#supplementary-information)
Nmut: Number of somatic exome mutation per genome
FLOH: Fraction of loss of heterozygosity per genome
NtAI: Number of telomeric allelic imbalances per genome
mBRCA: BRCA1 or BRCA2 mutation
NA: not applicable

What is claimed is:

1. A method for determining the prognosis of a subject with ovarian cancer, the method comprising
obtaining a cell sample from the subject;
determining the number of mutations in the exons of the tumor sample to determine a tumor mutation burden in the cell sample; and
determining whether the BRCA1 gene or BRCA2 gene is mutant or wild-type in the cells to determine a BRCA1 and BRCA2 status for the subject,
wherein a high tumor mutation burden and a mutation in either a BRCA1 gene or BRCA2 gene indicates the subject has a better prognosis than a subject with a low tumor mutation burden.

2. The method of claim 1, wherein the tumor mutation burden is compared to a reference tumor mutation burden sample for a subject population whose prognostic status is known.

3. The method of claim 1, wherein the ovarian cancer is a serous ovarian cancer.

4. The method of claim 3, wherein the serous ovarian cancer is high grade serous cancer.

5. The method of claim 1, wherein the cell sample contains or is suspected of containing ovarian cancer cells.

6. The method of claim 1, wherein a high tumor mutation burden indicates a longer progression-free survival (PFS).

7. The method of claim 1, wherein a high tumor mutation burden indicates a longer overall survival (OS).

8. The method of claim 6, wherein a high tumor mutation burden indicates a longer overall survival (OS).

9. The method of claim 1, wherein the total mutation burden comprises single-base substitution mutations.

10. The method of claim 1, wherein the method comprises determining the BRCA1 status of the subject.

11. The method of claim 1, wherein the method comprises determining the BRCA2 status of the subject.

12. The method of claim 10, wherein the method comprises determining the BRCA2 status of the subject.

13. The method of claim 1, wherein the BRCA1 mutation or BRCA2 mutation is a truncating mutation.

14. The method of claim 1, wherein the subject has had surgery to remove an ovarian tumor.

15. The method of claim 1, wherein the subject is classified as having a high tumor mutation burden at an Nmut of 60 or higher.

16. The method of claim 1, further comprising creating a record indicating the subject is likely to respond to the treatment for a longer or shorter duration of time based on the BRCA1 or BRCA2 genotype and total mutation burden.

17. The method of claim 16, wherein the record is created on a tangible medium.

18. A method for determining the prognosis of a subject who has had surgery to remove an ovarian tumor, the method comprising
obtaining a cell sample from the subject;
determining the tumor mutation burden in the cell sample;
determining whether the BRCA1 gene or BRCA2 gene is mutant or wild-type in the cells to determine a BRCA1 and BRCA2 status for the subject, and
using the comparison to determine the prognosis of the ovarian cancer, wherein a high tumor mutation burden and a mutation in either a BRCA1 gene or BRCA2 gene indicates the subject has a better prognosis than a subject with a low tumor mutation burden.

19. A method of diagnosing a sub-type of ovarian cancer, the method comprising
obtaining a cell sample from the subject;
determining the tumor mutation burden of cells in the tissue sample;
determining whether the BRCA1 gene or BRCA2 gene is mutant or wild-type in the cells to determine a BRCA1 and BRCA2 status for the subject, and
classifying the ovarian cancer as a serous ovarian cancer if the cell sample has a high tumor mutation burden and a mutation in either a BRCA1 gene or BRCA2 gene.
